(19) United States
(12) Patent Application Publication
(10) Pub. No.: US 2018/0085088 A1
(43) Pub. Date: Mar. 29, 2018

(54) ULTRASOUND FLOW IMAGING METHOD AND ULTRASOUND FLOW IMAGING SYSTEM

(71) Applicant: SHENZHEN MINDRAY BIO-MEDICAL ELECTRONICS CO., LTD., Shenzhen (CN)

(72) Inventors: Yigang DU, Shenzhen (CN); Rui FAN, Shenzhen (CN)

(21) Appl. No.: 15/827,991

(22) Filed: Nov. 30, 2017

Related U.S. Application Data
(63) Continuation of application No. PCT/CN2015/080934, filed on Jun. 5, 2015.

(51) Int. Cl. A61B 8/06; A61B 8/08; A61B 8/00

(52) U.S. Cl. CPC A61B 8/06; A61B 8/5223; A61B 8/0891; A61B 8/5246; A61B 8/462; A61B 8/488; A61B 8/463; A61B 8/5207; A61B 8/483; A61B 8/4466; A61B 8/085

(57) ABSTRACT

The present disclosure provides an ultrasound flow imaging method and ultrasound imaging system. The system may include a probe, a transmitting circuit which may excite the probe to transmit volume ultrasound beams to the scanning target, a receiving circuit 4, and a beam forming unit which may receive the echoes of the volume ultrasound beams and obtain the volume ultrasound echo signals, a data processing unit which may obtain the flow velocity vector information of the target point in the scanning target and the three-dimensional ultrasound image data based on the volume ultrasound echo signals, and a stereoscopic display device which may display the three-dimensional ultrasound image data to form the spatial stereoscopic image of the scanning target and superimposes the flow velocity vector information in the spatial stereoscopic image.

### TECHNICAL FIELD

The present disclosure relates to ultrasound flow imaging methods and systems, and more particularly to display technologies for flow imaging in an ultrasound imaging system.

### BACKGROUND

In a medical ultrasound imaging device, flow imaging is usually based on a two-dimensional image. Taking blood flow as an example, ultrasound waves are transmitted to an object to be examined, and the Doppler effect between red blood cells and the ultrasound waves are used by a color Doppler imaging device to obtain images, similar to pulsed wave Doppler imaging and continuous wave Doppler imaging. The color Doppler imaging device may include a two-dimensional display system, a pulsed wave Doppler (one-dimensional Doppler) blood flow analysis system, a continuous wave Doppler blood flow measurement system, and/or a color Doppler (two-dimensional Doppler) blood flow display system. An oscillator may generate two orthogonal signals between which the phase difference is π/2. The two orthogonal signals may respectively multiply the Doppler blood flow signal, and the products may be converted into digital signals by an A/D converter. After filtering by a comb filter to remove the low frequency components generated by vascular wall, valves or the like, the digital signals may be sent to an autocorrelator where autocorrelation may be performed. Since each sample includes the Doppler blood flow information generated by many red blood cells, the signals obtained by the autocorrelation are mixed signals of multiple blood flow velocities. The results of the autocorrelation may be sent to a velocity calculator and a variance calculator to obtain average velocities, which may be stored in a digital scan converter (DSC) together with blood flow spectrum information processed by FFT processing and two-dimensional image information. Finally, a color processor may perform a pseudo-color coding on the blood flow information based on the direction of the blood flow and the magnitude of the velocities, which may then be rendered on a color display.

In color Doppler blood flow imaging, only the magnitudes and directions of the velocities of the blood flow in the scanning plane are displayed. However, not only laminar flow exists in the blood flow. Rather, more complex flows, such as vortices or the like, may generally exists at arterial stenosis. Two-dimensional ultrasound scanning can only obtain the magnitudes and directions of the velocities of the blood flow in the scanning plane. With two-dimensional ultrasound imaging, the flow characteristics of the liquid in blood vessels or other tubular or liquid-contained organs cannot be realistically represented. In two-dimensional ultrasound imaging, only several isolated section images are displayed, or a pseudo three-dimensional image is obtained using several section images, which does not provide comprehensive and accurate information to the doctor. Therefore, it is desired to improve the existing flow imaging technologies to provide a more intuitive display solution for flow.

### SUMMARY

Accordingly, in order to overcome the drawbacks in the prior art, an ultrasound flow imaging method and ultrasound imaging system may be provided, which can provide more intuitive display for blood flow information and provide a better observation perspective for the user.

In some embodiments, an ultrasound flow imaging method may include: transmitting volume ultrasound beams to a scanning target; receiving echoes of the volume ultrasound beams and obtaining volume ultrasound echo signals; obtaining three-dimensional ultrasound image data of at least a part of the scanning target based on the volume ultrasound echo signals; obtaining flow velocity vector information of a target point in the scanning target based on the volume ultrasound echo signals; and displaying the three-dimensional ultrasound image data to form a spatial stereoscopic image of the scanning target and superimposing the flow velocity vector information in the spatial stereoscopic image.

In some embodiments, an ultrasound flow imaging system may include: a probe; a transmitting circuit which excites the probe to transmit volume ultrasound beams to a scanning target; a receiving circuit and a beam forming unit which receive echoes of the volume ultrasound beams and obtain volume ultrasound echo signals; a data processing unit which obtains three-dimensional ultrasound image data of at least a part of the scanning target based on the volume ultrasound echo signals and obtains flow velocity vector information of a target point in the scanning target based on the volume ultrasound echo signals; and a stereoscopic display device which receives the three-dimensional ultrasound image data and the flow velocity vector information of the target point, displays the three-dimensional ultrasound image data to form a spatial stereoscopic image of the scanning target, and superimposes the flow velocity vector information in the spatial stereoscopic image.

The present disclosure provides an ultrasound flow imaging method and system which can display the movement of the fluid in a spatial stereoscopic image, thereby providing more observation perspectives for the observer.

### BRIEF DESCRIPTION OF THE DRAWINGS

FIG. 1 is a block diagram of an ultrasound imaging system;

FIG. 2 schematically shows a plane ultrasound beam being transmitted;

FIG. 3 schematically shows a steered plane ultrasound beam;

FIG. 4 schematically shows a focused ultrasound beam;

FIG. 5 schematically shows a diffusion ultrasound;

FIG. 6A schematically shows the transducers of a two-dimensional array probe;

FIG. 6B schematically shows a three-dimensional scanning in an ultrasound propagation direction using the two-dimensional array probe;

FIG. 6C schematically shows the way for measuring the relative steering of the scanning body in FIG. 6B;

FIG. 7A schematically shows the divide of the two-dimensional array probe;

FIG. 7B schematically shows the transmission of the volume focused ultrasound beams;

FIG. 8 is a flow chart;

FIG. 9 is a flow chart;

FIG. 10 is a flow chart;

FIG. 11 schematically shows an image;

FIG. 12 schematically shows an image in which the stereoscopic cursor is superimposed;

FIG. 13A schematically shows the calculation of the flow velocity vector information in a first mode;

FIG. 13B schematically shows the calculation of the flow velocity vector information in a second mode;

FIG. 14A schematically shows the transmissions in two ultrasound propagation directions;

FIG. 14B schematically shows the synthesis of the flow velocity vector information based on FIG. 14A;

FIG. 15 schematically shows the stereoscopic display device;

FIG. 16 schematically shows the stereoscopic display device;

FIG. 17 schematically shows the stereoscopic display;

FIG. 18 schematically shows an image based on the first mode;

FIG. 19 schematically shows an image based on the second mode;

FIG. 20 schematically shows an image;

FIG. 21 schematically shows an image with the cloudy cluster block regions;

FIG. 22 schematically shows an image in which the target points are selected to form the trajectory;

FIG. 23 schematically shows a human-machine interaction; and

FIG. 24 schematically shows a cloudy cluster block region which has been rendered in color.

### DETAILED DESCRIPTION

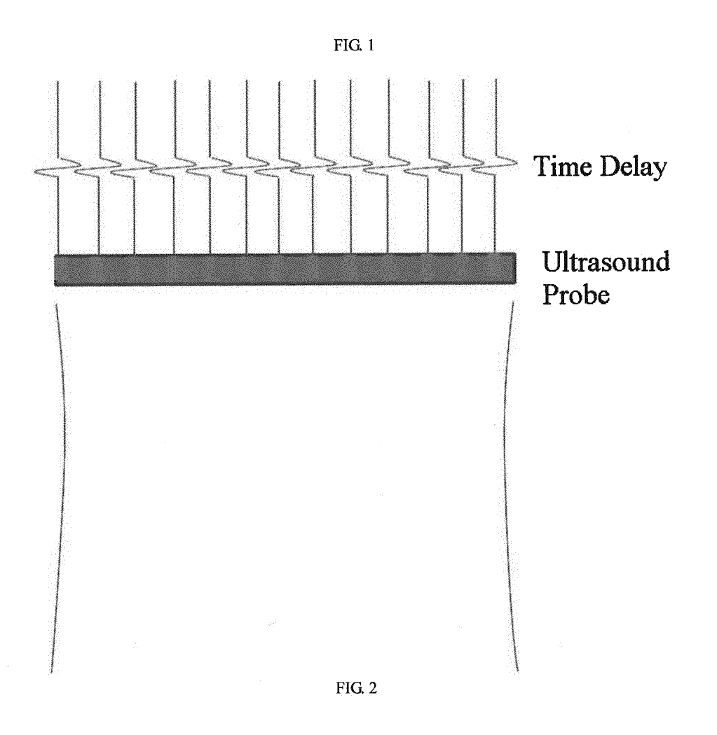

FIG. 1 schematically shows a block diagram of an ultrasound imaging system according to an embodiment of the present disclosure. As shown in FIG. 1, the ultrasound imaging system may generally include a probe 1, a transmitting circuit 2, a transmitting/receiving switch 3, a receiving circuit 4, a beam-forming unit 5, a signal processing unit 6, an image processing unit 7 and a stereoscopic display device 8.

In an ultrasound imaging process, the transmitting circuit 2 may transmit transmitting pulses, which have been delay focused and have certain amplitude and polarity, to the probe 1 through the transmitting/receiving switch 3. The probe 1 may be excited by the transmitting pulses and thereby transmit ultrasound waves to a scanning target (for example, organs, tissue, blood vessels or the like within a human or animal body, not shown), receive ultrasound echoes which are reflected by a target region and carry information related to the scanning target after a certain time interval, and convert the ultrasound echoes into electric signals. The receiving circuit may receive the electric signals converted by the probe 1 to obtain volume ultrasound echo signals and send the volume ultrasound echo signals to the beam-forming unit 5. The beam-forming unit 5 may perform processing such as a focus delaying, a weighting and a channel summing, etc. on the volume ultrasound echo signals and then send the volume ultrasound echo signals to the signal processing unit 6, where related signal processing procedures will be performed.

The volume ultrasound echo signals processed by the signal processing unit 6 may be sent to the image processing unit 7, where the signals may be processed in different ways according to the imaging mode desired by the user in order to obtain image data in different mode, such as two-dimensional image data and three-dimensional image data. Then, the image data may undergo the processing such as a logarithmic compression, a dynamic range adjustment and a digital scan conversion, etc. to form ultrasound image data of different modes, for example, including two-dimensional image data such as B images, C images or D images, etc., and three-dimensional ultrasound image data which can be sent to the display device for three-dimensional or spatial stereoscopic display.

The three-dimensional ultrasound image data generated by the image processing unit 7 may be sent to the stereoscopic display device 8 for display to form a spatial stereoscopic image of the scanning target. The spatial stereoscopic image herein may refer to a real three-dimensional image displayed in a physical space based on holographic display technologies or volume three-dimensional display technologies, including single frame image or multiple-frame images.

The probe 1 may generally include an array of multiple transducers. Each time the ultrasound waves are transmitted, all or a part of the transducers of the probe 1 may be used. In this case, each or each part of the used transducers may be respectively excited by the transmitting pulse and respectively transmit ultrasound wave. The ultrasound waves transmitted by the transducers may superpose with each other during the propagation thereof such that a resultant ultrasound beam transmitted to the scanning target can be formed. The direction of the resultant ultrasound beam may be the “ultrasound propagation direction” mentioned in the present disclosure.

The used transducers may be simultaneously excited by the transmitting pulses. Alternatively, a certain time delay may exist between the excitation times of the used transducers by the transmitting pulses. By controlling the time delay between the excitation times of the used transducers by the transmitting pulses, the propagation direction of the resultant ultrasound beam can be changed, as described in detail below.

By controlling the time delay between the excitation times of the used transducers by the transmitting pulses, it may also be possible that the ultrasound waves transmitted by the used transducers neither focus nor completely diffuse during the propagation thereof, but form a plane wave which is substantially planar as a whole. In the present disclosure, such plane wave without a focus may be referred to as a “plane ultrasound beam.”

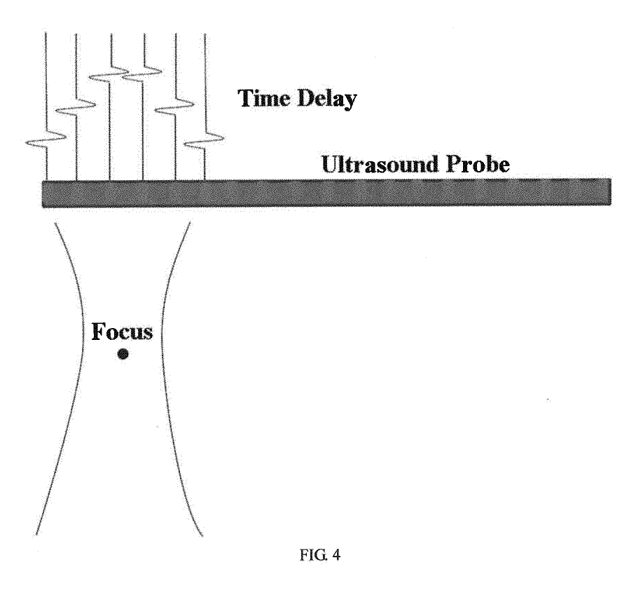

Alternatively, by controlling the time delay between the excitation times of the used transducers by the transmitting pulses, it may also be possible that the ultrasound waves transmitted by the transducers are superposed at a predetermined position such that the strength of the ultrasound waves at the predetermined position is maximum, in other words, such that the ultrasound waves transmitted by the transducers may be “focused” at the predetermined position. Such predetermined position may be referred to as a “focus.” In this case, the obtained resultant ultrasound beam may be a beam focused at the focus, which may be referred to as a “focused ultrasound beam” in the present disclosure. For example, FIG. 4 schematically shows the transmitting of a focused ultrasound beam. Here, the used transducers (in FIG. 4, only a part of the transducers of the probe 1 are used) may work with a predetermined transmission time delay (i.e., a predetermined time delay may exist between the excitation times of the used transducers by the transmitting pulses) and the ultrasound waves transmitted by the transducers may be focused at the focus to form the focused ultrasound beam.

Alternatively, by controlling the time delay between the excitation times of the used transducers by the transmitting pulses, it may also be possible that the ultrasound waves transmitted by the used transducers are diffused during the propagation to form a diffused wave which is substantially diffused as a whole. In the present disclosure, such diffused ultrasound wave may be referred to as a “diffused ultrasound beam.” An example of the diffused ultrasound beam is shown in FIG. 5.

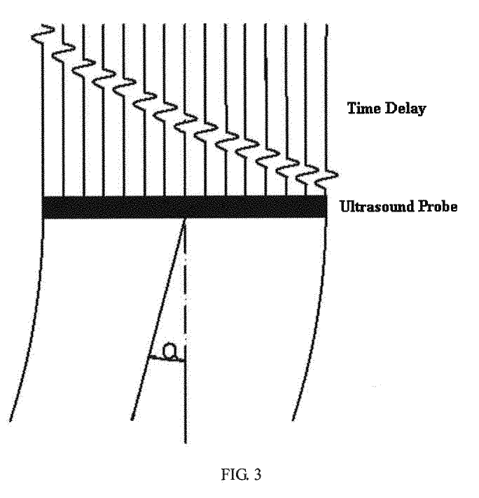

In the case that multiple transducers linearly arranged are excited simultaneously by electronic pulses, the transducers will simultaneously transmit ultrasound waves and the propagation direction of the resultant ultrasound beam will be the same as the normal direction of the plane on which the transducers are arranged. For example, for the plane beam vertically transmitted shown in FIG. 2, there is no time delay between the used transducers (i.e. there is no time delay between the excitation times of the transducers by the transmitting pulses) and the transducers are excited simultaneously. The ultrasound beam formed thereby is a plane beam, i.e. a plane ultrasound beam. The propagation direction of this plane ultrasound beam is substantially perpendicular to the surface of the probe 1 from which the ultrasound waves are transmitted, i.e. the angle between the propagation direction of the resultant ultrasound beam and the normal direction of the plane on which the transducers are arranged is zero degree. However, in the case that there is time delay between the excitation pulses applied to the transducers, the transducers will successively transmit ultrasound waves according to the time delay, and there will be an certain angle between the propagation direction of the resultant ultrasound beam and the normal direction of the plane on which the transducers are arranged. This angle is the steered angle of the resultant beam. By changing the time delay, the magnitude of the steered angle, and the direction of the steering in the scanning plane of the resultant beam with respect to the normal direction of the plane on which the transducers are arranged, of the resultant beam may be adjusted. For example, FIG. 3 schematically shows a plane beam with a steered angle. In this case, there is a predetermined time delay between the used transducers (i.e., between the excitation times of the used transducers by the transmitting pulses), and the transducers are excited in a predetermined order by the transmitting pulses. The ultrasound beam generated thereby is a plane beam, i.e. a plane ultrasound beam, and there is an angle (for example, the angle α in FIG. 3) between the propagation direction of this plane ultrasound beam and the normal direction of the plane on which the transducers of the probe 1 are arranged. This angle is the steered angle of the plane ultrasound beam and may be adjusted by changing the time delay.

Similarly, regardless of the plane ultrasound beam, the focused ultrasound beam or the diffused ultrasound beam, the “steered angle” of the resultant beam formed between the direction of the resultant beam and the normal direction of the plane on which the transducers are arranged can be adjusted by adjusting the time delay between the excitation times of the used transducers by the transmitting pulses. The “resultant beam” herein may be the plane ultrasound beam, the focused ultrasound beam or the diffused ultrasound beam mentioned above.

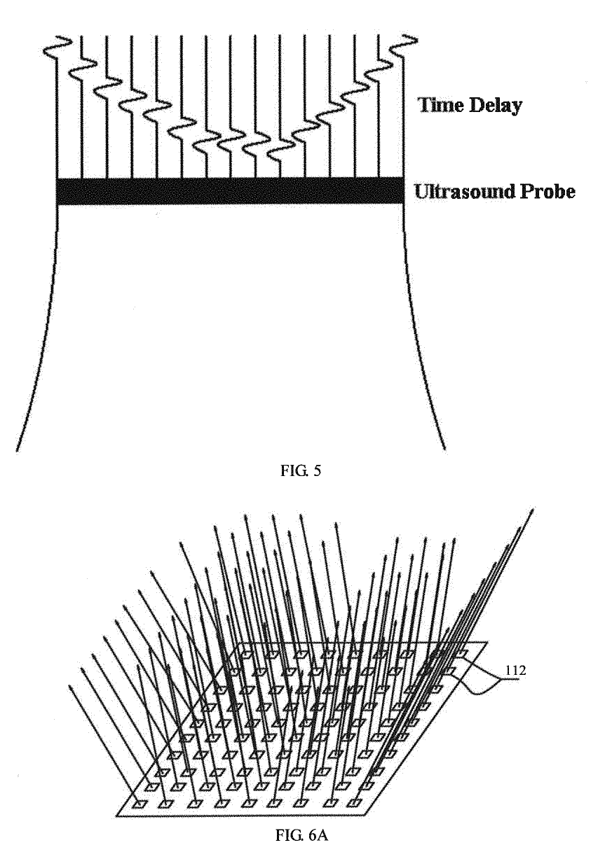

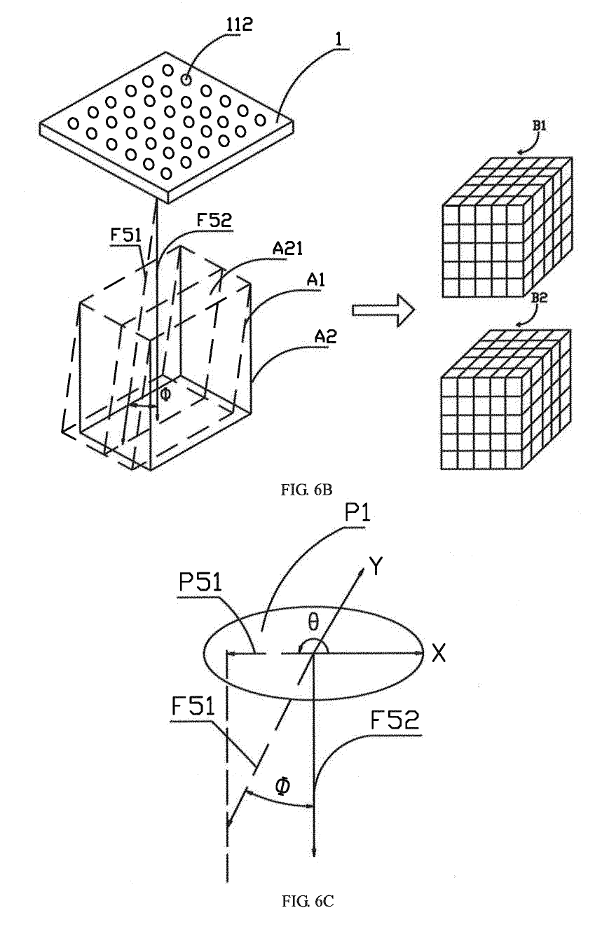

When performing three-dimensional ultrasound imaging, a two-dimensional array probe may be used, as shown in FIG. 6A. The two-dimensional array probe may include multiple transducers 112, which are arranged in transverse and longitudinal directions. Each transducer of the two-dimensional array probe may be provided with a delay control line which may be used to control the time delay of the corresponding transducer. During the transmitting and the receiving of the ultrasound beam, the beam control and the dynamic focus of the ultrasound beam may be implemented by adjusting the time delay of each transducer, thereby changing the direction of the beam in order to implement the scanning of the beam in a three-dimensional space to obtain three-dimensional image data. As shown in FIG. 6B, the two-dimensional array probe 1 may include multiple transducers 112. By changing the time delay of the transducers used in current transmitting of the ultrasound waves, the transmitted ultrasound beam may propagate in the direction indicated by the dot-chain arrow F51 and form a scanning body A1 (the three-dimensional structure drawn by the dot-chain lines in FIG. 6B) for obtaining three-dimensional image data in the three-dimensional space. The scanning body A1 may have a predetermined steering with respect to a reference body A2 (the three-dimensional structure drawn by the solid lines in FIG. 6B). The reference body A2 herein may be formed in the three-dimensional space by making the ultrasound beam transmitted by the used transducers to propagate in the normal direction of the plane on which the transducers are arranged (indicated by the solid-line arrow F52). Therefore, the steering amount of the scanning body A1 with respect to the reference body A2 may be used to measure the steered angle in a three-dimensional space of a scanning body formed by the propagation of an ultrasound beam in a certain direction with respect to the reference body A2. In the present disclosure, the steering amount may be measured by following two angles: the predetermined steered angle Φ between the propagation direction of the ultrasound beam and the normal direction of the plane on which the transducers are arranged in the scanning plane A21 (the quadrilateral drawn by the dot-chain lines in FIG. 6B) of the ultrasound beam in the scanning body, which is in the range of [0, 90°), and the rotation angle θ formed by, in the plane rectangular coordinate system in the plane P1 on which the transducers are arranged, counterclockwise rotating from X axis to the projection P51 (the dot-chain arrow in plane P1 in FIG. 6C) of the propagation direction of the ultrasound beam on the plane P1 on which the transducers are arranged, which is in the range of [0, 360°). In the case that the steered angle Φ zero, the steering amount of the scanning body A1 with respect to the reference body A2 is zero. During three-dimensional ultrasound imaging, by changing the time delay of each transducer, the magnitude of the steered angle Φ and the rotation angle θ may be changed to change the steering amount of the scanning body A1 with respect to the reference body A2, thereby forming different scanning bodies in different ultrasound propagation directions in the three-dimensional space. The transmitting of the scanning bodies above may also be achieved using a probe group formed by arranging linear probes in an array, and the transmitting mode may be the same. For example, as shown in FIG. 6B, three-dimensional ultrasound image data B1 may be obtained from the volume ultrasound echo signals returned from the scanning body A1, and three-dimensional ultrasound image data B2 may be obtained from the volume ultrasound echo signals returned from the scanning body A2.

In the present disclosure, ultrasound beams which “are transmitted to a scanning target, propagate in a space in which the scanning target is located, and are used to form the scanning body above” may be regarded as a volume ultrasound beam, which may include the group of ultrasound beams transmitted one or more times. Therefore, based on the type of the ultrasound beam, the plane ultrasound beams which “are transmitted to a scanning target, propagate in a space in which the scanning target is located, and are used to form the scanning body above” may be regarded as a volume plane ultrasound beam, the focused ultrasound beams which “are transmitted to a scanning target, propagate in a space in which the scanning target is located, and are used to form the scanning body above” may be regarded as a volume focused ultrasound beam, and the diffused ultrasound beams which “are transmitted to a scanning target, propagate in a space in which the scanning target is located, and are used to form the scanning body above” may be regarded as a volume diffused ultrasound beam, etc. The volume ultrasound beam may include the volume plane ultrasound beam, the volume focused ultrasound beam, the volume diffused ultrasound beam, and so on. The name of the type of the ultrasound beam may be added between the “volume” and the “ultrasound beam.”

The volume plane ultrasound beam may generally almost cover the entire imaging area of the probe 1. In the case of performing ultrasound imaging using the volume plane ultrasound beam, one frame of three-dimensional ultrasound image (the one frame of ultrasound image herein should be understood as including one frame of two-dimensional image data or one frame of three-dimensional image data, and the same below) may be obtained by one transmitting, therefore the imaging frame rate may be very high. While in the case of performing ultrasound imaging using the volume focused ultrasound beam, since the beam is focused at the focus, only one or several scan lines can be obtained by each transmission, therefore multiple transmissions need to be performed to obtain all scan lines within the imaging area so as to obtain one frame of three-dimensional ultrasound image of the imaging area by combining all scan lines. Therefore, in the case of performing ultrasound imaging using the volume focused ultrasound beam, the frame rate is relatively low. However, the energy of the volume focused ultrasound beam is concentrated and the image data is only obtained at the energy concentrated location. Accordingly, the signal to noise ratio of the obtained echo signals is high and the ultrasound images with better quality can be obtained.

Based on ultrasound three-dimensional imaging, the system of the present disclosure may display the real ultrasound stereoscopic images and the velocity vectors of the flow in a superimposed manner. Therefore, the user can not only have a better viewing angle, but also a view the flow information, such as the velocities of the blood and the flow directions, etc. at the location being scanned in real time. Furthermore, the images can represent the path of travel of the flowing fluid more realistically. The fluid herein may include blood, intestinal fluid, lymph, tissue fluid, cell fluid or other body fluids. Various embodiments of the present disclosure will be described in details with reference to the drawings.

As shown in FIG. 8, the present disclosure may provide an ultrasound flow imaging method, which may be based on three-dimensional imaging and realistically represent the ultrasound image in a stereoscopic space using spatial stereoscopic display technologies, thereby providing better viewing angle for the user. Therefore, the user can view the ultrasound stereoscopic images represented realistically from multiple angles, and thereby can know the location being scanned in real time. Furthermore, the flow information may be represented more realistically by the images, thereby providing more comprehensive and more accurate image analysis results.

As shown in FIG. 8, an ultrasound flow imaging method provided by an embodiment may include steps S100 to S5500 below. In step S100, the transmitting circuit 2 may excite the probe 1 to transmit volume ultrasound beams to the scanning target such that the volume ultrasound beams propagate in the space in which the scanning target is located to form scanning bodies as shown in FIG. 6. In some embodiments, the probe 1 may be a two-dimensional array probe, or may also be a probe group formed by arranging linear probes in an array, etc. Using the two-dimensional array probe or the probe groups in array, the echo data of one scanning body may be timely obtained by one scanning, and thereby the scanning speed and the imaging speed may be increased.

In the present disclosure, the volume ultrasound beams transmitted to the scanning target may include at least one of volume focused ultrasound beam, volume non-focused ultrasound beam, volume virtual source ultrasound beam, volume non-diffractive ultrasound beam, volume diffused ultrasound beam, volume plane ultrasound beam and other type of beam, or include the combination thereof including at least more than two types of beams (“more than” herein may include the number following this phrase itself, and the same below). Of course, the embodiments of the present disclosure will not be limited to the volume ultrasound beams mentioned above.

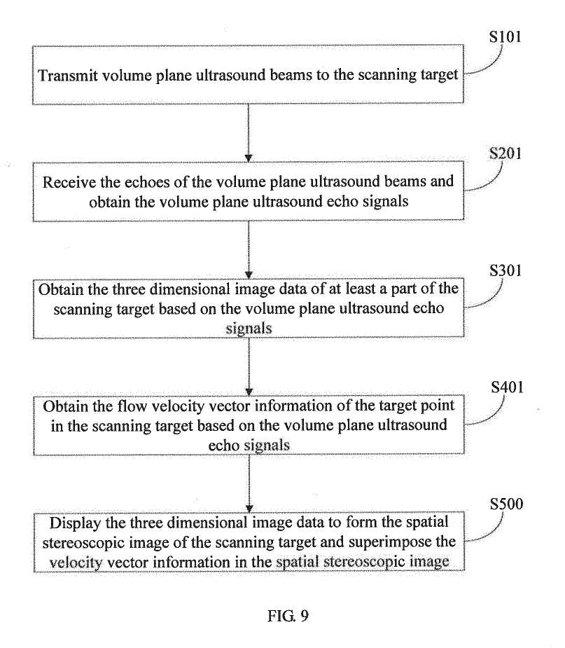

In some embodiments, as shown in FIG. 9, the volume plane waves may be used, which may save the scanning time of the three-dimensional ultrasound image and increase the frame rate of the imaging, thereby achieving high-frame rate flow-velocity-vector imaging. Therefore, the step S100 may include a step S101 in which volume plane ultrasound beams may be transmitted to a scanning target. In step 201, the echoes of the volume plane ultrasound beams may be received, thereby obtaining volume plane ultrasound echo signals which may be used to reconstruct three-dimensional ultrasound image data and/or calculate the velocity vectors of the flow at target points in the scanning target. For example, in step 301 of FIG. 9, three-dimensional ultrasound image data of at least a part of the scanning target may be obtained based on the volume plane ultrasound echo signals; and in step S401, the velocity vectors of the flow at the target points in the scanning target may be obtained based on the volume plane ultrasound echo signals.

The scanning target may be the tubular tissue in which fluid flows in a human or animal body, such as organs, tissues, vessels or the like. The target points in the scanning target may be the points or locations of interest in the scanning target, which may generally be represented as, in the spatial stereoscopic image of the scanning target displayed by the stereoscopic display device, spatial points or spatial locations of interest which can be marked or displayed. A spatial point or spatial locations may be one spatial point or a spatial neighborhood of one spatial point, and the same below.

Alternatively, in step S100, the volume focused ultrasound beams may be transmitted to the scanning target such that the volume focused ultrasound beams propagate in the space in which the scanning target is located to form the scanning body. Thereby, in step S200, the echoes of the volume focused ultrasound beams may be received to obtain the volume focused ultrasound echo signals which may be used to reconstruct the three-dimensional ultrasound image data and/or calculate the velocity vectors of the flow at the target points in the scanning target.

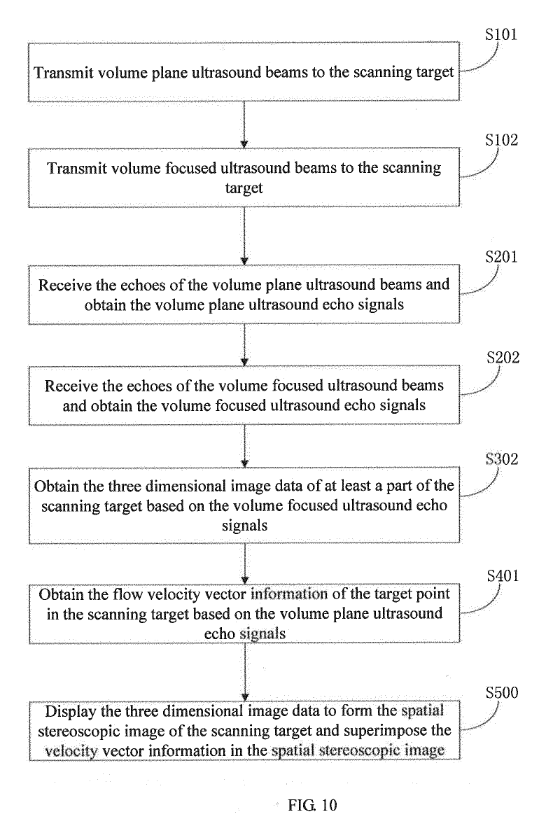

Alternatively, as shown in FIG. 10, step S100 may include step S101 and step S102. In step S101, volume plane ultrasound beams may be transmitted to the scanning target; in step 201, the echoes of the volume plane ultrasound beams may be received to obtain volume plane ultrasound echo signals; and in step S401, the velocity vectors of the flow at the target points in the scanning target may be obtained based on the volume plane ultrasound echo signals. In step S102, volume focused ultrasound beams may be transmitted to the scanning target; in step 202, the echoes of the focused ultrasound beams may be received to obtain focused volume ultrasound echo signals; and in step S302, three-dimensional ultrasound image data of at least a part of the scanning target may be obtained based on the volume focused ultrasound echo signals. The volume focused ultrasound echo signals may be used to reconstruct high quality three-dimensional ultrasound image data in order to obtain three-dimensional ultrasound image data with better quality as background image.

In the case that two types of volume ultrasound beams are used in step S100, the two types of volume ultrasound beams may be transmitted to the scanning target alternately. For example, the processes for transmitting the volume focused ultrasound beams to the scanning target may be inserted between the processes for transmitting the volume plane ultrasound beams to the scanning body. I.e. the step S101 and the step S102 shown in FIG. 10 may be performed alternately. This way, the synchronization between the acquisition of the two types of volume ultrasound beam image data may be ensured, and the accuracy of superimposing the flow velocity vectors at the target points on the background image may be increased.

In step S100, the volume ultrasound beams may be transmitted to the scanning target based on Doppler imaging technologies in order to obtain the volume ultrasound echo signals for calculating the flow velocity vectors at the target points. For example, the volume ultrasound beams may be transmitted to the scanning target in one ultrasound propagation direction such that the volume ultrasound beams propagate in the space in which the scanning target is located to form a scanning body. Then, the three-dimensional ultrasound image data used for calculating the flow velocity vectors at the target points may be obtained based on the volume ultrasound echo signals returned from the one scanning body.

Of course, in order to enable the calculated results of the flow velocity vectors at the target points to represent the velocity vectors at the target points in the real three-dimensional space more realistically, In some embodiments, the volume ultrasound beams may be transmitted to the scanning target in multiple ultrasound propagation directions, where each scanning body may be derived from the volume ultrasound beams transmitted in one ultrasound propagation direction. The volume ultrasound echo signals returned from the Multiple scanning bodies may be used to obtain the image data used for calculating the flow velocity vectors at the target points. For example, the step S200 and the step S400 may include:

first, receiving echoes from the ultrasound beams of the multiple scanning bodies to obtain multiple groups of echo signals;

then, one velocity component at the target point in the scanning target may be calculated based one of the multiple groups of echo signals, thereby respectively obtaining multiple velocity components based on the multiple groups of echo signals; and

synthesizing the velocity vector at the target point based on the multiple velocity components to obtain flow velocity vector information at the target point.

The multiple ultrasound propagation directions may include two or more ultrasound propagation directions.

During the transmitting of the ultrasound beams to the scanning target in multiple ultrasound propagation directions, the transmitting of the ultrasound beams to the scanning target in different ultrasound propagation directions may be performed alternately. For example, in the case that the volume ultrasound beams are transmitted to the scanning target in two ultrasound propagation directions, the volume ultrasound beams may be transmitted to the scanning target first in a first ultrasound propagation direction first, and then in a second ultrasound propagation, thereby achieving one scan cycle. Then, the scan cycle may be repeated sequentially. Or, the volume ultrasound beams may be transmitted to the scanning target first in one ultrasound propagation direction, and then in another ultrasound propagation direction, and so on, until the transmitting in all ultrasound propagation directions are performed. The different ultrasound propagation directions may be achieved by changing the time delay of each or each part of the transducers to be used in the transmitting of the ultrasound waves, which may be specifically understood with reference to the description with regard to FIG. 2 to FIG. 6A-6C.

For example, the process of transmitting the volume plane ultrasound beams to the scanning target in multiple ultrasound propagation directions may include: transmitting, to the scanning target, a first volume ultrasound beam which has a first ultrasound propagation direction; and transmitting, to the scanning target, a second volume ultrasound beam which has a second ultrasound propagation direction. The echoes of the first volume ultrasound beam and the echoes of the second volume ultrasound beam may be received respectively to obtain first volume ultrasound echo signals and second ultrasound echo signals. Two velocity components may be obtained based on the two groups of ultrasound echo signals. The flow velocity vector at the target point may be obtained by synthesizing the two velocity components. The arrangement with regard to the ultrasound propagation direction may refer to the detailed description above with respect to FIG. 2. In some of the embodiments, the first volume ultrasound beam and the second volume ultrasound beam may be plane ultrasound beams, and correspondingly the first volume ultrasound echo signals and the second volume ultrasound echo signals may be first volume plane ultrasound echo signals and second volume plane ultrasound echo signals.

As another example, the process of transmitting the volume plane ultrasound beams to the scanning target in multiple ultrasound propagation directions may also include: transmitting the volume plane ultrasound beams to the scanning target in N (N is a natural number greater than or equal to 3) ultrasound propagation directions and receiving the echoes thereof to obtain N (N is a natural number greater than or equal to 3) groups of volume ultrasound echo signals each of which may be derived from the volume ultrasound beams transmitted in one ultrasound propagation direction. The N groups of volume ultrasound echo signals may be used to calculate the flow velocity vectors at the target points.

In addition, In some embodiments, a portion or all of the transducers may be excited to transmit the volume ultrasound beam to the scanning target in one or more ultrasound propagation directions. The volume ultrasound beams in the present embodiment may be, for example, volume plane ultrasound beam.

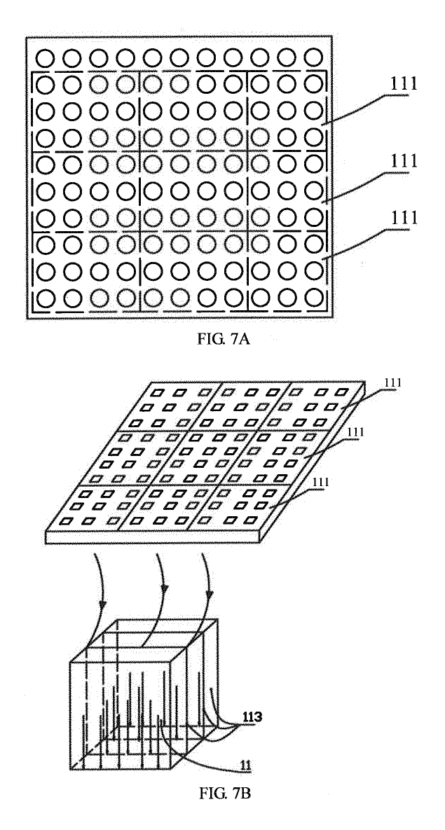

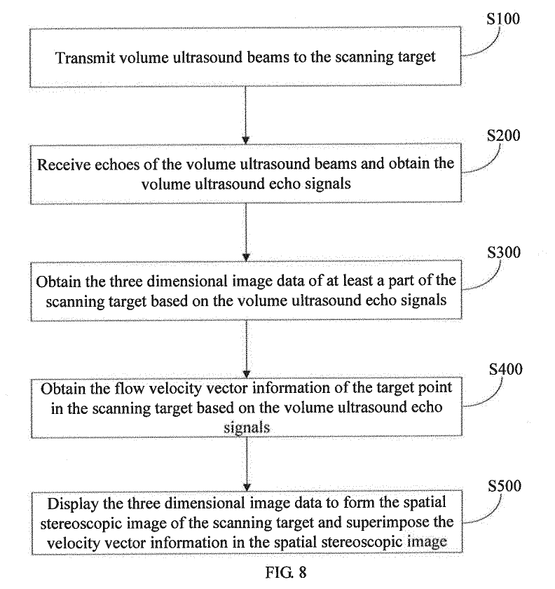

As another example, in some of the embodiments of the present disclosure, as shown in FIG. 7A and FIG. 7B, the transducers may be divided into multiple transducer regions 111. A part or all of the transducer regions may be excited to transmit volume ultrasound beams to the scanning target in one or more ultrasound propagation direction, where each scanning body may be derived from the volume ultrasound beams transmitted in one ultrasound propagation direction. The formation of the scanning bodies may refer to the detailed description with regard to FIG. 6A-FIG. 6C above, and will not be described again. The volume ultrasound beams in the present embodiment may, for example, include, but not limited to, one of volume focused ultrasound beam, volume plane ultrasound beam, etc. In the case that the volume focused ultrasound beams are used in the present embodiment, after the transducers are divided into multiple transducer regions, one focused ultrasound beam may be generated by exciting one of the transducer regions, and multiple focused ultrasound beams may be generated by exciting multiple transducer regions, thereby forming the volume focused ultrasound beams to obtain one scanning body. As shown in FIG. 7A and FIG. 7B, taking transmitting of the focused ultrasound beams as an example, each transducer region 111 may be used to generate at least one focused ultrasound beam (the arc with arrow in the figure). When multiple transducer regions 111 are excited to generate focused ultrasound beams, multiple focused ultrasound beams may propagate in the space in which the scanning target is located to form a scanning body 11 formed by volume focused ultrasound beams. The focused ultrasound beams in the scanning body 11 which are located in a same plane may form a scanning plane 113 (represented by the solid arrows in the figure, each solid arrow representing one focused ultrasound beam), and the scanning body 11 may also be regarded as being formed by multiple scanning planes 113. By changing the time delay of the transducers used in the transmitting of the ultrasound waves in each transducer region 111, the direction of the focused ultrasound beam may be changed, thereby changing the propagation directions of the multiple focused ultrasound beams in the space in which the scanning target is located.

In some embodiments, the volume ultrasound beams may be transmitted to the scanning target in each ultrasound propagation direction for multiple times to obtain multiple volume ultrasound echo signals for subsequent ultrasound image data processing. For example, the volume plane ultrasound beams may be transmitted to the scanning target respectively in multiple ultrasound propagation directions for multiple times, or the volume focused ultrasound beams may be transmitted to the scanning target respectively in one or more ultrasound propagation directions for multiple times. Each transmission of the volume ultrasound beams may correspondingly obtain one volume ultrasound echo signals.

The multiple transmitting of the volume ultrasound beams to the scanning target in different ultrasound propagation directions may be performed alternately, which enables the echo data obtained to be used to calculate the velocity vectors at the target points at substantially the same time in order to increase the calculation accuracy of the flow velocity vectors. For example, in the case that the volume ultrasound beams are respectively transmitted to the scanning target in three ultrasound propagation directions for N times, the volume ultrasound beams may first be transmitted to the scanning target in a first ultrasound propagation direction for at least one time, and then be transmitted to the scanning target in a second ultrasound propagation direction for at least one time, and then be transmitted to the scanning target in a third ultrasound propagation direction for at least one time, thereby achieving one scanning cycle. Finally, the scanning cycle above may be repeated sequentially until the transmitting in all of the ultrasound propagation directions is completed. In each scanning cycle, the number of the transmitting of the volume ultrasound beams in different ultrasound propagation directions may be the same, or may also be different with each other For example, in the case that the volume ultrasound beams are transmitted in two ultrasound propagation directions, the order of the transmitting may be A1 B1 A2 B2 A3 B3 A4 B4 . . . Ai Bi, and so on, where Ai represents the i-th transmitting in the first ultrasound propagation direction and Bi represents the i-th transmitting in the second ultrasound propagation direction. In the case that the volume ultrasound beams are transmitted in three ultrasound propagation directions, the order of the transmitting may be A1 B1 B1C1 A2 B2 B2C2 A3 B3 B3C3 . . . Ai Bi Bi Ci, and so on, where Ai represents the i-th transmitting in the first ultrasound propagation direction, Bi represents the i-th transmitting in the second ultrasound propagation direction, and Ci represents the i-th transmitting in the third ultrasound propagation direction.

In addition, in the case that two kinds of ultrasound beams are transmitted to the scanning target in step S100 above, the two kinds of ultrasound beams may be transmitted alternately. For example, in some embodiments, the step S100 may include:

first, transmitting volume focused ultrasound beams to the scanning target for multiple times to obtain reconstructed three-dimensional ultrasound image data;

and then, transmitting volume plane ultrasound beams to the scanning target in one or more ultrasound propagation direction to obtain image data used for calculating the velocity vectors at the target points.

Accordingly, the processes of transmitting volume focused ultrasound beams to the scanning target may be inserted between the processes of transmitting volume plane ultrasound beams to the scanning target. For example, the multiple transmitting of the volume focused ultrasound beams to the scanning target may be evenly inserted between the multiple transmitting of the volume plane ultrasound beams.

For example, the successive transmitting of the volume plane ultrasound beams “Ai Bi Ci” above may be mainly used to obtain data used for calculating the velocity information at the target point, while transmitting of the other kind of volume ultrasound beams used for obtaining the reconstructed three-dimensional ultrasound image may be inserted between the successive transmitting “Ai Bi Ci.” The way for alternately transmitting two kinds of beams will be described in detail below taking inserting the transmitting of the volume focused ultrasound beams between the successive transmitting of the volume plane ultrasound beams “Ai Bi Ci” as an example.

The volume plane ultrasound beams may be transmitted to the scanning target respectively in three ultrasound propagation directions for multiple times according to the following order:

A1 B1 Cl D1A2 B2 C2 D2 A3 B3 C3 D3 . . . Ai Bi CiDi, and so on.

Where Ai represents the i-th transmitting in the first ultrasound propagation direction, Bi represents the i-th transmitting in the second ultrasound propagation direction, Ci represents the i-th transmitting in the third ultrasound propagation direction, and Di represents the i-th transmitting of the volume focused ultrasound beams.

The methods above provide relatively simple ways for inserting the transmitting of the volume focused ultrasound beams. In addition, the transmitting of the volume focused ultrasound beam may be inserted for one time after the multiple transmitting of the volume plane ultrasound beams in different ultrasound propagation directions are completed, or, at least one portion of the multiple transmitting of the volume plane ultrasound beams to the scanning target and at least one portion of the multiple transmitting of the volume focused ultrasound beams to the scanning target may be performed alternately, etc. Besides, any method which can achieve alternately performing at least one portion of the multiple transmitting of the volume plane ultrasound beams to the scanning target and at least one portion of the multiple transmitting of the volume focused ultrasound beams to the scanning target may also be used. In the present embodiment, the volume focused ultrasound beams may be used to obtain better three-dimensional ultrasound image data, while the volume plane ultrasound beams may be used to obtain high real-time flow velocity vector information due to the high frame rate thereof. Furthermore, for better synchronization of the obtaining of the two kinds of data, the two kinds of ultrasound beams may be transmitted alternately.

Therefore, the order and the rules for performing the multiple transmitting of the volume ultrasound beams to the scanning target in different ultrasound propagation directions may be selected as needed, which will not be listed herein and not limited to the specific example provided above.

In step S200, the receiving circuit 4 and the beam-forming unit 5 may receive the echoes of the volume ultrasound beams transmitted in step S100 and obtain the volume ultrasound echo signals.

The type of the echoes of the volume ultrasound beams received and the volume ultrasound echo signals thereby generated may correspond to the type of the volume ultrasound beams transmitted in step S100. For example, in the case that the echoes of the volume focused ultrasound beams transmitted in step S100 are received, the volume focused ultrasound echo signals may be obtained; and in the case that the echoes of the volume plane ultrasound beams transmitted in step S100 are received, the volume plane ultrasound echo signals may be obtained; and so on. The name of the type of the ultrasound beams may be added between the “volume” and the “ultrasound echo signals.”

When the receiving circuit 4 and the beam-forming unit 5 receive the echoes of the volume ultrasound beams transmitted in step S100, the echoes of the volume ultrasound beams transmitted in step S100 may be received by each or each part of the transducers used in the transmitting of the ultrasound beams during the time-sharing transmitting and receiving; or, the transducers in the probe may be classified as receiving transducers and transmitting transducers, and each or each part of the receiving transducers may be used to receive the echoes of the volume ultrasound beams transmitted in step S100; etc. The receiving of the volume ultrasound beams and the obtaining of the volume ultrasound echo signals may be similar to those in the art.

When the volume ultrasound beams are transmitted in each of the ultrasound propagation directions in step S100, the echoes of the volume ultrasound beams may be received in step S200 to obtain a group of volume ultrasound echo signals. For example, when the echoes of the volume ultrasound beams transmitted to the scanning target in one ultrasound propagation direction in step S100 are received, a group of volume ultrasound echo signals may be obtained in step S200, and correspondingly the three-dimensional ultrasound image data of at least a part of the scanning target and the flow velocity vector information at the target points may be respectively obtained in step S300 and the step S400 based on the group of volume ultrasound echo signals. When the echoes of the volume ultrasound beams transmitted to the scanning target in multiple ultrasound propagation directions are received in step S200, multiple groups of volume ultrasound echo signals may be obtained, each of which may be derived from the echoes of the volume ultrasound beams transmitted in one ultrasound propagation direction. Then, correspondingly, in step S300 and the step S400, the three-dimensional ultrasound image data of at least a part of the scanning target may be obtained based on one of the multiple groups of volume ultrasound echo signals, and the flow velocity vector information at the target points may be obtained based on the multiple groups of volume ultrasound echo signals.

In addition, in the case that the volume ultrasound beams are transmitted in each of the ultrasound propagation directions for multiple times, the group of volume ultrasound echo signals obtained by receiving the echoes of the volume ultrasound beams in step S200 may include multiple ultrasound echo signals, where each of the ultrasound echo signals may be obtained by transmitting the ultrasound beams for one time.

For example, in the case that the volume plane ultrasound beams are transmitted to the scanning target in multiple ultrasound propagation directions in step S100, the echoes of the corresponding volume plane ultrasound beams in the multiple ultrasound propagation directions may be respectively received in step S200 to obtain multiple groups of volume plane ultrasound echo signals. Each group of volume plane ultrasound echo signals may include multiple volume plane ultrasound echo signals, and each of the multiple volume plane ultrasound echo signals may be derived from the echoes obtained by transmitting the volume plane ultrasound beams to the scanning target in one ultrasound propagation direction for one time.

As another example, in the case that the volume focused ultrasound beams are transmitted to the scanning target for multiple times in step S100, the echoes of the volume focused ultrasound beams may be received in step S200 to obtain multiple groups of volume focused ultrasound echo signals.

Therefore, the type of the echoes of the volume ultrasound beams received in step S200 and the number of the groups of the corresponding volume ultrasound echo signals may correspond to the type and the number of the transmitting of the volume ultrasound beams transmitted in step S100.

In step S300, the image processing unit 7 may obtain the three-dimensional image data of at least a part of the scanning target based on the volume ultrasound echo signals. Using three-dimensional imaging based on the volume ultrasound echo signals, the three-dimensional image data B1 and B2 as shown in FIG. 6B may be obtained, which may include the location information of spatial points and corresponding image information of the spatial points. The image information may include grayscale, color or other characteristic information.

In some embodiments, the three-dimensional ultrasound image data may be obtained using the volume plane ultrasound beams, or may also be obtained using the volume focused ultrasound beams. However, since the energy of the volume focused ultrasound beam transmitted each time is more concentrated and the image data is obtained at the energy concentration position, the obtained echo signals may have high signal-to-noise ratio and the obtained three-dimensional ultrasound image data may have better quality. Furthermore, the volume focused ultrasound beams may have narrow main lobe and low side lobes, therefore the obtained three-dimensional ultrasound image data may have high lateral resolution. Therefore, in some embodiments, in step S500, the three-dimensional ultrasound image data may be obtained using the volume focused ultrasound beams. In addition, in order to obtain three-dimensional ultrasound image data with better quality, the volume focused ultrasound beams may be transmitted for multiple times in step S100 to obtain a frame of three-dimensional ultrasound image data.

Of course, the three-dimensional ultrasound image data may also be obtained based on the volume plane ultrasound echo signals obtained in step S200 above. In the case that multiple groups of volume ultrasound echo signals are obtained in step S200, one of the groups of volume ultrasound echo signals may be selected and used to obtain the three-dimensional ultrasound image data of at least a part of the scanning target.

In order to fully present the movement of the flow in spatial stereoscopic images, the step S300 may further include obtaining enhanced three-dimensional ultrasound image data of at least a part of the scanning target using grayscale blood flow imaging. The grayscale blood flow imaging may also be referred to as two-dimensional blood flow displaying, and is a new imaging method which may scan the blood flow, the blood vessels and the surrounding soft tissue using digital coded ultrasound technology and display the images in gray scale.

In the embodiments above, the processing to the three-dimensional ultrasound image data may be three-dimensional data processing performed on the whole three-dimensional ultrasound image data, or may also be a set of processing performed on one or more frames of two-dimensional ultrasound image data in one frame of three-dimensional ultrasound image data. Therefore, in some embodiments, the step S300 may include processing one or more frames of two-dimensional ultrasound image data in one frame of three-dimensional ultrasound image data using the grayscale blood flow imaging to obtained the enhanced three-dimensional ultrasound image data of the scanning target.

In step S400, the image processing unit 7 may obtain the flow velocity vector information at the target points in the scanning target based on the volume ultrasound echo signals obtained in step S200 above. The flow velocity vector information mentioned herein may include at least the velocity vectors (i.e. magnitude and direction of the velocity) at the target points, and may further include the location information of the target points in the spatial stereoscopic image. Of course, the flow velocity vector information may further include any other information related to the velocity at the target points which may be obtained based on the magnitude and direction of the velocity, such as acceleration information, etc.

For example, as shown in FIG. 11, a part of the spatial stereoscopic image of the scanning target formed by displaying the three-dimensional ultrasound image data above is shown. The object 210 and the object 220 may respectively represent two blood vessels within a human or animal, in which the overall flow directions of the blood flow are opposite, as shown by the arrows in the figure. In some embodiments, the target points may include one or more discrete spatial points located within the scanning target, or may respectively include neighborhood space range or data block of the one or more discrete spatial points, such as the range of the cone 211 or sphere 221 in FIG. 11.

As another example, in some embodiments, in step S400, a distribution density instruction inputted by the user may be obtained, target points may be selected randomly within the scanning target based on the distribution density instruction, and the flow velocity vector information at the selected target points may be calculated. The obtained flow velocity vector information may be marked on the background image (for example, the spatial stereoscopic image of the scanning target) for display on the stereoscopic display device. For example, for the object 210 and the object 220 in the part of the stereoscopic image in the FIG. 11, the user may input the distribution density of the target points to be arranged within the object 210 and the object 220 through human-machine interface device. In FIG. 11, the cone 210 and the sphere 220 may represent the selected target points. We can see from the figure that their distribution density within respective object 210 and object 220 are different. The distribution density herein may be spatial distribution density, such as possibility of the presence of the target points within a certain stereoscopic region. The certain stereoscopic region may be the whole or part of the stereoscopic region of the object 210 or the object 220 in the image of the scanning target. For example, in FIG. 11, the target points selected initially may be located at front section of the spatial region in which the object 210 or the object 220 is located in the overall flow direction. For example, the target points may be selected within a region 212 within the stereoscopic region in which the object 210 is located, or be selected within a region 222 within the stereoscopic region in which the object 220 is located. By selecting the distribution density of the target points within the parts of the stereoscopic region such as the region 212 and the region 222, etc., or by obtaining the distribution density information based on the locations of the target points within the parts of the stereoscopic region such as the region 212 and the region 222, etc., the distribution density instruction inputted by the user may be obtained.

Then, the flow velocity vectors at the selected target points may be calculated, thereby obtaining the flow velocity vector information at the selected target points. The obtained flow velocity vector information may be marked on the spatial stereoscopic images of the scanning target for display on the stereoscopic display device.

As another example, In some embodiments, the step S400 may further include:

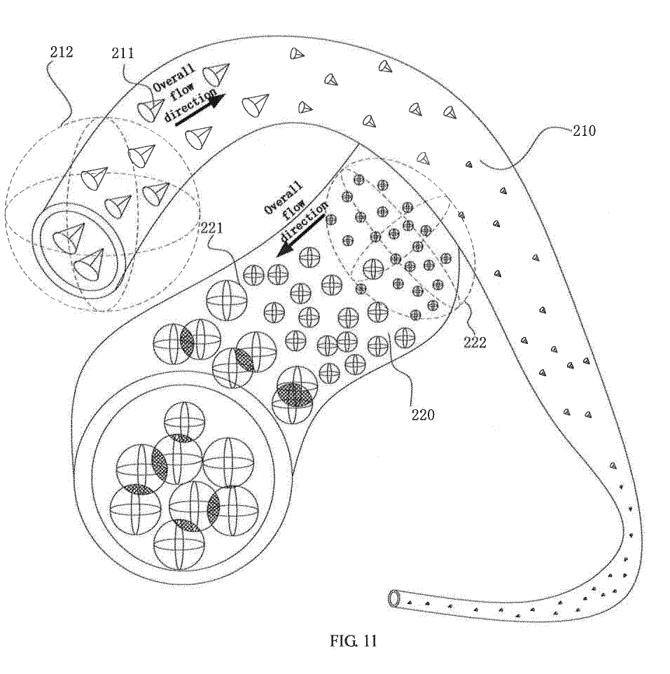

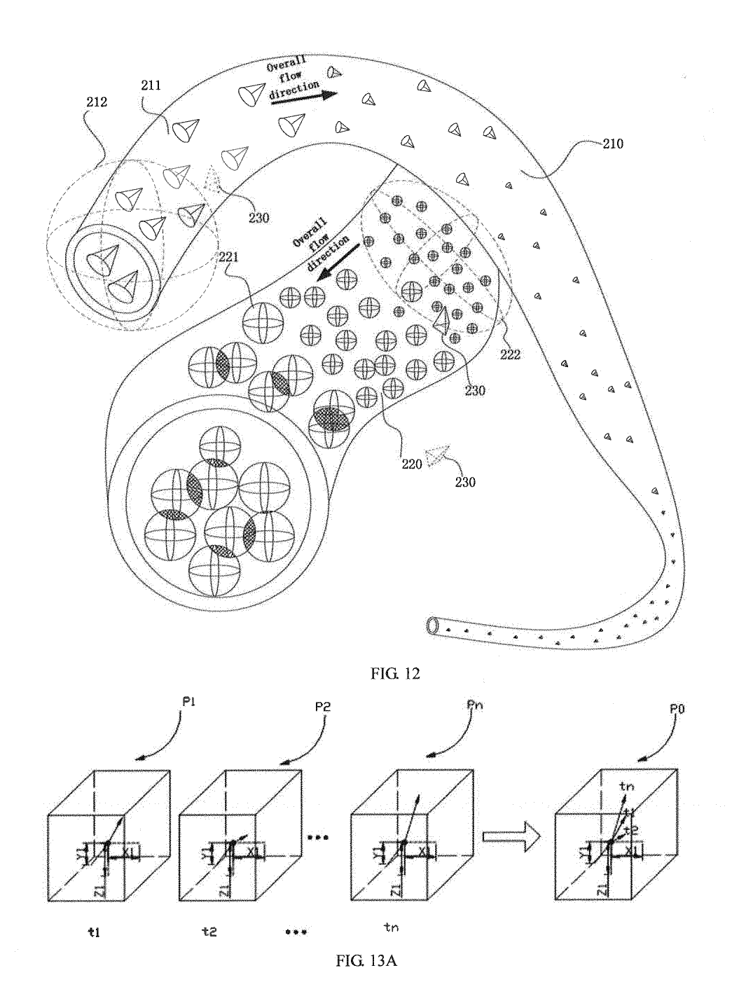

obtaining location marking instruction inputted by the user, obtaining the target points selected based on the location marking instruction, and calculating the flow velocity vector information at the selected target points. The obtained flow velocity vector information may be marked on the spatial stereoscopic images of the scanning target for display on the stereoscopic display device. For example, in FIG. 12, the locations to be marked may be selected by gesture input in the image region of the spatial stereoscopic image or by moving the location of the stereoscopic cursor 230 in the image region, thereby generating the location marking instruction. As shown in FIG. 12, the stereoscopic cursor 230 may be pyramid, and the pyramids drawn with different type of lines may represent the locations of the stereoscopic cursor 230 at different times. In addition, the stereoscopic cursor 230 may be used to select the target points within whole or part (212, 222) of the stereoscopic region of the object 210 or the object 220 in the image region of the scanning target.

In the present embodiment, the target points may be selected by the user, and the two specific examples above provide two ways for selecting the target points, including selecting the locations of the target points or selecting initial positions used for calculating the flow velocity vectors at the target points. However, the present disclosure is not limited thereto. For example, the locations of the target points or the initial locations used for calculating the flow velocity vectors at the target points may be selected randomly in the scanning target based on the distribution density preset by the system. This way, the user may be provided with flexible selection methods, thereby increasing the user experience. Furthermore, based on the two methods for interacting with the user above, the distribution density instructions or the location marking instructions inputted by the user may be obtained by selecting the distribution density or the locations of target points through moving the stereoscopic cursor 230 displayed in the spatial stereoscopic images or through gestures. The configuration of the stereoscopic cursor 230 is not limited, and any configuration having stereoscopic sense of vision may be used. Furthermore, The stereoscopic cursor 230 may be distinguished from other marks used for marking the flow velocity vector information at the target points and from the background images (such as the images of tissue) using colors or shapes.

The process of obtaining the flow velocity vector information at the target points in the scanning target based on the volume ultrasound echo signals in step S400 will be described in detail below.

The flow velocity vector information obtained in step S400 may be mainly used to be superimposed on the spatial stereoscopic images. Therefore, based on different methods for displaying the flow velocity vector information, different flow velocity vector information may be obtained in step S400.

For example, in some embodiments, the step S400 may include calculating flow velocity vectors of the target point at a first display position in three-dimensional ultrasound image data at different times based on the volume ultrasound echo signals obtained in step S200 to obtain flow velocity vector information at the target point in the three-dimensional ultrasound image data at different times. Thereby, in subsequent step S500, the flow velocity vector information at the first location at the various times may be displayed on the spatial stereoscopic images. As shown in FIG. 13A, the three-dimensional ultrasound image data P1, P2 . . . Pn at time t1, t2 . . . to may be respectively obtained based on the volume ultrasound echo signals obtained in step S200, and then the flow velocity vectors of the target point at the first display positions (the positions of the black spheres in the figure) in the spatial stereoscopic images at the various times. In the present disclosure, the first display positions of the target point in the spatial stereoscopic images at the various times may be always located at the spatial position (X1, Y1, Z1) in the three-dimensional image data. Therefore, during the superimposed display of the flow velocity vectors in the subsequent step S500, the flow velocity vectors calculated at different times may be displayed at the position (X1, Y1, Z1) in the spatial stereoscopic image P0 displayed by the stereoscopic display device. In the case that, with reference to the embodiments above, a part or all of the target points are selected by the user or selected by the system by default, the corresponding first display positions may be obtained correspondingly, and the flow velocity vector information at the first display position in the three-dimensional image data at the current time may be calculated for the superimposed display. Such display mode may be referred to as the first mode in the present disclosure, and the same below. FIG. 13A schematically shows the display effect of the spatial stereoscopic image P0.

In others embodiments of the present disclosure, the step S400 may include calculating flow velocity vectors successively generated continuous movement of the target point to corresponding positions in the spatial stereoscopic image based on the volume ultrasound echo signals obtained in step S200, thereby obtaining the flow velocity vector information of the target point. In the present embodiment, the corresponding flow velocity vectors at various corresponding positions during the continuous movement of the target point from the initial position may be obtained by successively calculating the flow velocity vector of the target moving from one position to another position in the spatial stereoscopic image in a time interval. That is, the calculation positions for determining the flow velocity vectors in the spatial stereoscopic image of the present embodiment may be obtained by calculation. Then, in step S500 below, what is displayed in superimposed manner may be the flow velocity vector information at the positions in the spatial stereoscopic image obtained by calculation at various times.

As shown in FIG. 13B, the three-dimensional ultrasound image data P11, P12 . . . Pln corresponding to time t1, t2 . . . to may be respectively obtained based on the volume ultrasound echo signals obtained in step S200. Then, the initial position of the target point may be determined based on part or all of the target points selected by the user or the distribution density of the target points selected by the system by default as described in the embodiments above, such as the first point at (X1, Y1, Z1) in FIG. 13B. Then, the flow velocity vector (indicated by the arrow in P11) at the initial position in the three-dimensional ultrasound image data P11 at time t1 may be calculated. After that, the position (X2, Y2, Z2) in the three-dimensional ultrasound image data P12 at time t2 to which the target point (i.e. the black dot in the figure) is moved from the initial position in the three-dimensional ultrasound image data P11 at time t1 may be calculated, and then, the flow velocity vector at the position (X2, Y2, Z2) in the three-dimensional ultrasound image data P12 may be obtained based on the volume ultrasound echo signals. The obtained flow velocity vector may be superimposed on the spatial stereoscopic image. For example, a displacement in a time interval to the second time t2 (the time interval=t2−t1) in the direction of the flow velocity vector at the position (X1, Y1, Z1) in the three-dimensional ultrasound image data P11 at time t1 may be calculated, thereby obtaining the second display position of the target point at the first time t1 in the second time three-dimensional ultrasound image data, and then the flow velocity vector at the second display position may be obtained based on the volume ultrasound echo signals obtained in step S200 above, thereby obtaining the flow velocity vector information of the target point in the three-dimensional ultrasound image data P12 at time t2, and so on. For each two adjacent times, the displacement in the time interval between the two adjacent times in the direction of the flow velocity vector of the target point corresponding to the first time may be obtained and the corresponding position of the target point in the three-dimensional ultrasound image data at the second time may be determined based on the displacement, and then, the flow velocity vector of the target point with which the target point is moved from the ultrasound image at the first time to the ultrasound image at the second time may be obtained based on the volume ultrasound echo signals. This way, the blood flow velocity vector information with which the target point is continuously moved from (XI, Y1, Z1) to (Xn, Yn, Zn) in the three-dimensional ultrasound image data may be obtained. Thereby, the flow velocity vectors with which the target point is continuously moved from the initial position to the corresponding positions in the spatial stereoscopic image at different times may be obtained to obtain the flow velocity vector information of the target point which may be superimposed on the spatial stereoscopic image P10 for display.

In the display method of the present embodiment, the displacement of the target point in the time interval may be calculated, and the corresponding position of the target point in the three-dimensional ultrasound image data may be determined based on the displacement. The target point may be moved in the time interval starting from the position selected initially. The time interval may be determined based on the transmission frequency of the system, or based on display frame rate. Or, the time interval may also be inputted by the user. The position which the target point achieves after the movement may be calculated based on the time interval inputted by the user, and then the flow velocity vector information at such position may be obtained for display. Initially, N initial target points may be marked on the image as the methods shown in FIG. 11 and FIG. 12. For each initial target point, a flow velocity vector mark may be set to represent the magnitude and direction of the flow velocity at this point, as shown in FIG. 13B. In step S500 for superimposed display, the flow velocity vectors correspondingly obtained when the target point is continuously moved to corresponding positions in the spatial stereoscopic image may be marked to generate velocity vector mark which is flowing over time, as shown in FIG. 11 and FIG. 12 (in which the flow velocity vector marks are cones and spheres, respectively). The flow velocity vector information may be obtained by marking on FIG. 13B, therefore, with the change of time, the arrow of each original target point will change in position in the newly generated spatial stereoscopic image P10. This way, the movement of the velocity vector marks such as stereoscopic arrows may be used to generate a visible flowing process, such that the user can see approximate real flow flowing, such as the flowing process of the blood in blood vessels. Such display mode may be referred to as the second mode in the present disclosure, and the same below. Similarly, FIG. 13B schematically shows the display effect of the spatial stereoscopic image P10.

Based on part or all of the target points selected by the user or by the system by default and the transmission form of the volume ultrasound beams in step S100, in the embodiments above, the following methods may be used to obtain the flow velocity vectors of the target points in the scanning target at the corresponding positions in the three-dimensional ultrasound image data at any time.

In the first method, one group of ultrasound echo signals obtained by transmitting the volume ultrasound beams in one ultrasound propagation direction in step S100 may be used to calculate the flow velocity vector information of the blood flow in the scanning target. In this process, the flow velocity vector of the target point at the corresponding position in the spatial stereoscopic image may be obtained by calculating the displacement and the movement direction of the target point in a preset time interval.

As described above, in the present embodiment, the volume plane ultrasound echo signals may be used to calculate the flow velocity vector information of the target point. Therefore, in some embodiments, the displacement and direction of the movement of the target point in the scanning target in the preset time interval may be calculated based on one group of volume plane ultrasound echo signals.

In the present embodiment, speckle tracking may be used to calculate the flow velocity vectors of the target point at the corresponding position in the spatial stereoscopic image. Alternatively, Doppler ultrasound imaging may be used to obtain the flow velocity vector of the target point in an ultrasound propagation direction. And alternatively, the velocity vector components of the target point may be obtained based on the time gradient and the spatial gradient at the target point.

For example, in some embodiments, obtaining the flow velocity vectors of the target point in the scanning target at the corresponding position in the spatial stereoscopic image based on the volume ultrasound echo signals may include following steps.

First, at least two frames of three-dimensional ultrasound image data may be obtained based on the obtained volume ultrasound echo signals. For example, at least a first frame of three-dimensional ultrasound image data and a second frame of three-dimensional ultrasound image data may be obtained.

As described above, in the present embodiment, the volume plane ultrasound beams may be used to obtain the image data used for calculating the flow velocity vectors of the target point. The volume plane ultrasound beams may substantially propagate in the entire imaging area. Therefore, one frame of three-dimensional ultrasound image data may be obtained by transmitting a group of volume plane ultrasound beams which have the same angle using a two dimensional array probe, receiving the echoes and performing three-dimensional imaging process. In case that the frame rate is 10000, i.e. 10000 transmissions per second, 10000 frames of three-dimensional ultrasound image data may be obtained in each second. In the present disclosure, the three-dimensional ultrasound image data of the scanning target obtained by processing the volume plane beam echo signals of the volume plane ultrasound beams may be referred to as “volume plane beam echo image data.”

Thereafter, a three-dimensional tracking area may be selected in the first frame of three-dimensional ultrasound image data. The three-dimensional tracking area may contain the target points of which the velocity vectors are desired to be obtained. In one embodiment, the three-dimensional tracking area may be a three-dimensional area with any shape centered at the target point, such as a cube area.

Then, a three-dimensional area corresponding to the three-dimensional tracking area may be searched out from the second frame of three-dimensional ultrasound image data. For example, a three-dimensional area which has maximum similarity with the three-dimensional tracking area may be searched out as a tracking result area. The measurement of the similarity herein may be common measurements in the art.

At last, the velocity vectors of the target point may be obtained based on the positions of the three-dimensional tracking area and the tracking result area above and the time interval between the first and second frame of three-dimensional ultrasound image data. For example, the magnitude of the flow velocity vector may be obtained by dividing the distance between the three-dimensional tracking area and the tracking result area (i.e. the displacement of the target point within the preset time interval) by the time interval between the first and second frame of volume plane beam echo image data, and the direction of the flow velocity vector may be the direction of a line extending from the three-dimensional tracking area to the tracking result area, i.e. the moving direction of the target point within the preset time interval.

In order to increase the accuracy of the calculation of the flow velocity vector using the speckle tracking, wall filtering may be performed on each frame of three-dimensional ultrasound image data, i.e., the wall filtering may be performed in the time direction for each spatial point in the three-dimensional ultrasound image data. The signals representing the tissue in the three-dimensional ultrasound image data have small changes over time, while the signals representing the flow such as the blood flow have large changes. Therefore, a high-pass filter may be used as the wall filter for the flow signals such as the signals representing the blood flow. After the wall filtering, the signals representing the flow with high frequency are retained, while the signals representing the tissue with low frequency are filtered out. In the wall-filtered signals, the signal to noise ratio of the signals representing the flow is greatly increased, which helps to increase the accuracy of the calculation of the flow velocity vector. In the present embodiment, the wall filtering performed on the obtained three-dimensional ultrasound image data may also be suitable for other embodiments.

In one embodiment, obtaining the velocity vector of the target point based on the time gradient and the spatial gradient at the target point may include following steps.

First, at least two frames of three-dimensional ultrasound image data may be obtained based on the volume ultrasound echo signals. Alternatively, the wall filtering may additionally be performed on the three-dimensional ultrasound image data.

Then, the gradient in the time direction of the target point may be obtained based on the three-dimensional ultrasound image data, and a first velocity component of the target point in the ultrasound propagation direction may be obtained based on the three-dimensional ultrasound image data.

Thereafter, a second velocity component in a first direction and a third velocity component in a second direction at the target point may be obtained based on the gradient and the first velocity component, where the first direction, the second direction and the ultrasound propagation direction are perpendicular to each other.

Finally, the first velocity component, the second velocity component and the third velocity component may be synthesized to obtain the flow velocity vector of the target point.

In the present embodiment, the first direction, the second direction and the ultrasound propagation direction are perpendicular to each other, which may be considered as a three-dimensional coordinate system in which the ultrasound propagation direction is one of the coordinate axes. For example, the ultrasound propagation direction may be Z axis, and the first direction and the second direction may be X axis and Y axis.

Assuming that the wall-filtered three-dimensional ultrasound image data is represented as P(x(t),y(t),z(t)), the formula (1) may be obtained according to the chain rule by finding the derivative of P along the time direction:

$$\frac{dP ( x ( t ) , y ( t ) , z ( t ) )}{dt} = \frac{\partial P}{\partial x} \frac{dx}{dt} + \frac{\partial P}{\partial y} \frac{dy}{dt} + \frac{\partial P}{\partial z} \frac{dz}{dt} \qquad ( 1 )$$

The second velocity component of the flow in X direction is represented as

$${v}_{x} = \frac{dx}{dt} ,$$

the third velocity component in Y direction is represented as

$${v}_{y} = \frac{dy}{dt} ,$$

and the first velocity component in Z direction is represented as

$${v}_{z} = \frac{dz}{dt} .$$

Accordingly, the formula (1) may be transformed into formula (2):

$$\frac{dP ( x ( t ) , y ( t ) , z ( t ) )}{dt} = \frac{\partial P}{\partial x} {v}_{x} + \frac{\partial P}{\partial y} {v}_{y} + \frac{\partial P}{\partial z} {v}_{z} \qquad ( 2 )$$

Where

$$\frac{\partial P}{\partial x} , \frac{\partial P}{\partial y} , \frac{\partial P}{\partial z}$$

may be obtained by calculating the gradients of the three-dimensional ultrasound image data in X, Y and Z direction, respectively, and

$$\frac{dP ( x ( t ) , y ( t ) , z ( t ) )}{dt}$$

may be obtained by calculating, for each spatial point in the three-dimensional ultrasound image data, the gradient in the time direction based on multiple frames of three-dimensional ultrasound image data.

Thereafter, using the least squares solution, the formula (2) may be transformed into a linear regression equation formula (3):

$$[ \begin{matrix}{P}_{1}^{t} \\ {P}_{2}^{i} \\ ⋮ \\ {P}_{N}^{t}\end{matrix} ] = [ \begin{matrix}{p}_{1}^{x} & {p}_{1}^{y} & {p}_{1}^{z} \\ {p}_{2}^{x} & {p}_{2}^{y} & {p}_{2}^{z} \\ ⋮ & ⋮ & ⋮ \\ {p}_{N}^{x} & {p}_{N}^{y} & {p}_{N}^{z}\end{matrix} ] [ \begin{matrix}{v}_{x} \\ {v}_{y} \\ {v}_{z}\end{matrix} ] + [ \begin{matrix}{ɛ}_{1} \\ {ɛ}_{2} \\ ⋮ \\ {ɛ}_{N}\end{matrix} ] \qquad ( 3 )$$

Where the subscript i in

$${P}_{i}^{t} = \frac{{dP}_{i} ( x ( t ) , y ( t ) , z ( t ) )}{dt} , {P}_{i}^{x} = \frac{\partial {P}_{i}}{\partial x} , {p}_{i}^{y} = \frac{\partial {P}_{i}}{\partial y} and {P}_{i}^{z} = \frac{\partial {P}_{i}}{\partial z}$$

may represent the i^{th} calculation of the gradient of the three-dimensional ultrasound image data in X, Y and Z directions. The gradients of the spatial points in the three coordinate axes calculated in multiple times may form a parameter matrix A. It is assumed that the gradients are calculated for N times, and it is also assumed that the flow velocity remains constant for this period of time since the time taken by the N calculations is very short. εᵢ represents random error. Herein, the formula (3) satisfies Gauss-Markov theorem, and its solution is the formula (4) below:

$$[ \begin{matrix}{v}_{x} \\ {v}_{y} \\ {v}_{z}\end{matrix} ] = {( {A}^{T} A )}^{- 1} {A}^{T} u \qquad ( 4 )$$

Where the parameter

$$A = [ \begin{matrix}{P}_{1}^{x} & {P}_{1}^{y} & {P}_{1}^{z} \\ {P}_{2}^{x} & {P}_{2}^{y} & {P}_{2}^{z} \\ ⋮ & ⋮ & ⋮ \\ {P}_{N}^{x} & {P}_{N}^{y} & {P}_{N}^{z}\end{matrix} ] , and u = [ \begin{matrix}{P}_{1}^{t} \\ {P}_{2}^{t} \\ ⋮ \\ {P}_{N}^{t}\end{matrix} ] .$$

According to the Gauss-Markov theorem, the variance of the random error εᵢ may be represented as the formula (5) below:

var(εᵢ)=σ_{A}² (5)

Based on the relationship model of the gradient, the velocity values V_{Z} and the average thereof at each spatial point in the ultrasound propagation direction (i.e. Z direction) at different times may be obtained according to Doppler ultrasound measurement, and the variance of the random error and the parameter matrix at each spatial point in the ultrasound propagation direction may be calculated. V_{D} is a group of velocity value at different times obtained by Doppler ultrasound measurement, and v_{Z} in the formula (6) is the average obtained by the Doppler ultrasound measurement.

$${V}_{D} - B [ \begin{matrix}{v}_{x} \\ {v}_{y} \\ {v}_{z}\end{matrix} ] + {ɛ}_{j} \\ Where \\ {V}_{D} = [ \begin{matrix}{v}_{1} \\ {v}_{2} \\ ⋮ \\ {v}_{N}\end{matrix} ] , and B = [ \begin{matrix}0 & 0 & 1 \\ 0 & 0 & 1 \\ ⋮ & ⋮ & ⋮ \\ 0 & 0 & 1\end{matrix} ] . \qquad ( 6 )$$

The variance of the random error εᵢ based on the formula (3) may be represented as the formula (7) below.

var(εⱼ)−σ_{B}² (7)

Two different variances may be calculated using the formula (5) and (7). The formula (3) above may be solved using weighted least squares method utilizing the variance of the random error and the parameter matrix at each spatial point in the ultrasound propagation direction as known information, as shown by the formula (8) below.

$${( w [ \begin{matrix}A \\ B\end{matrix} ] )}^{T} ( w [ \begin{matrix}A \\ B\end{matrix} ] ) [ \begin{matrix}{v}_{x} \\ {v}_{y} \\ {v}_{z}\end{matrix} ] = {( w [ \begin{matrix}A \\ B\end{matrix} ] )}^{T} [ \begin{matrix}u \\ {V}_{D}\end{matrix} ] \qquad ( 8 )$$

Where the weighting factor

$$w = ⌊ \begin{matrix}{I}_{A} \sqrt{\frac{1}{{σ}_{A}^{2}}} & 0 \\ 0 & {I}_{B} \sqrt{\frac{1}{{σ}_{B}^{2}}}\end{matrix} ⌋ ,$$

O is zero matrix, and I_{A} and I_{B} are unit matrixes, the orders of which respectively correspond to the numbers of rows of the matrix A and matrix B. The weighting factor may be the square root of the reciprocal of the variance of the random error in the linear error equation.

After three velocities vₓ, v_{y} and v_{z} which are perpendicular to each other are obtained, the magnitude and direction of the vector flow velocity by three-dimensional spatial fitting.

In one embodiment, Doppler ultrasound imaging may be used to obtain the flow velocity vector of the target point, as described below.

In the Doppler ultrasound imaging method, the ultrasound beams may be successively transmitted to the scanning target multiple times in an ultrasound propagation direction. The echoes of the transmitted ultrasound beams may be received to obtain multiple volume ultrasound echo signals. Each value in each volume ultrasound echo signal may correspond to a value at one target point when scanning the scanning target in an ultrasound propagation direction. Step S400 may include following steps.

A Hilbert transform along the ultrasound propagation direction or an IQ demodulation may be performed on the multiple volume ultrasound echo signals. After the beamforming, multiple three-dimensional ultrasound image data may be obtained, which may represent the value at each target point using complex number. After N transmissions and receptions, there are N complex numbers at each target point which vary over time. Thereafter, the magnitude of the velocity of a target point z in the ultrasound propagation direction may be calculated according to the following two formulas:

$$\begin{matrix} {v}_{z} = - \frac{c}{4 π {f}_{0} {T}_{prf}} arctan ( \frac{ \{ R ( 1 ) \}}{ \{ R ( 1 ) \}} ) & formula ( 9 ) \\ R ( 1 ) = \frac{1}{N - 1} {\sum }_{i = 0}^{N \text{?} 2} x ( i ) x ( i + 1 ) + y ( i ) y ( i + 1 ) + j [ y ( i + 1 ) x ( i ) - x ( i + 1 ) y ( i ) ] \\ \text{?} \text{indicates text missing or illegible when filed} & formula ( 10 )\end{matrix}$$

Where Y is the calculated velocity value in the ultrasound propagation direction, c is velocity of sound, f₀ is the center frequency of the probe, T_{prf} is the time interval between two transmissions, N is the number of the transmission, x(i) is the real part corresponding to the i^{th} transmission, y(i) is the imaginary part corresponding to the i^{th} transmission, [symbol] is the imaginary part operator, and [symbol] is the real part operator. The formula above may be used to calculate the flow velocity at a fixed position.

Similarly, the magnitude of the flow velocity vector at each target point may be calculated using the N complex numbers.

The direction of the flow velocity vector may be the ultrasound propagation direction, i.e. the ultrasound propagation direction corresponding to the multiple volume ultrasound echo signals.

Generally, in ultrasound imaging, the moving velocity of the scanning target, or of the moving part thereof, may be obtained by performing Doppler processes on the volume ultrasound echo signals based on Doppler principle. For example, after the volume ultrasound echo signals are obtained, the moving velocity of the scanning target, or of the moving part thereof, may be obtained based on the volume ultrasound echo signals using autocorrelation estimation or cross correlation estimation. The method for Doppler-processing the volume ultrasound echo signals to obtain the moving velocity of the scanning target, or of the moving part thereof, may be any method being or to be used by which the moving velocity of the scanning target, or of the moving part thereof, may be calculated based on the volume ultrasound echo signals, and will not be described in detail.

Of course, for the volume ultrasound echo signals corresponding to an ultrasound propagation direction, it will not be limited to the two methods above. Other methods known or to be used in the art may also be used.

In the second method, multiple groups of volume ultrasound echo signals may be obtained by transmitting the volume ultrasound beams in multiple ultrasound propagation directions in step S100 and receiving the echoes of the volume ultrasound beams from multiple scanning bodies. The multiple groups of volume ultrasound echo signals may be used to calculate the flow velocity vector information of the target point in the scanning target. In this process, one velocity vector component at the position in the spatial stereoscopic image corresponding to the target point in the scanning target may be calculated based on one group of volume ultrasound echo signals of the multiple groups of volume ultrasound echo signals, and accordingly multiple velocity vector components at the corresponding position may be obtained based on the multiple groups of volume ultrasound echo signals. And then, the flow velocity vector at the corresponding position of the target point in the spatial stereoscopic image may be synthesized based on the multiple velocity vector components.

As described above, in the present embodiment, the volume plane ultrasound echo signals may be used to calculate the flow velocity vector of the target point. Therefore, in one embodiment, one velocity vector component of the target point in the scanning target at one position may be calculated based on one group of volume plane ultrasound echo signals of multiple groups of volume plane ultrasound echo signals, and accordingly multiple velocity vector components at such position may be obtained based on the multiple groups of volume plane ultrasound echo signals.

In the present embodiment, the methods for calculating one velocity vector component of the target point in the scanning target based on one of the multiple groups of volume ultrasound echo signals may be similar to those in the first method. For example, the velocity vector component of the target point at corresponding position may be obtained by calculating the displacement and moving direction of the target point in a preset time interval based on one group of volume ultrasound echo signals. In the present embodiment, the speckle tracking as described above may be used to calculate the velocity vector component of the target point. Alternatively, Doppler ultrasound imaging may also be used to obtain the velocity vector component of the target point in an ultrasound propagation direction. Alternatively, the blood flow velocity vector component of the target point may be obtained based on the time gradient and the spatial gradient at the target point. Reference may be made to the detailed description of the first method above for details.

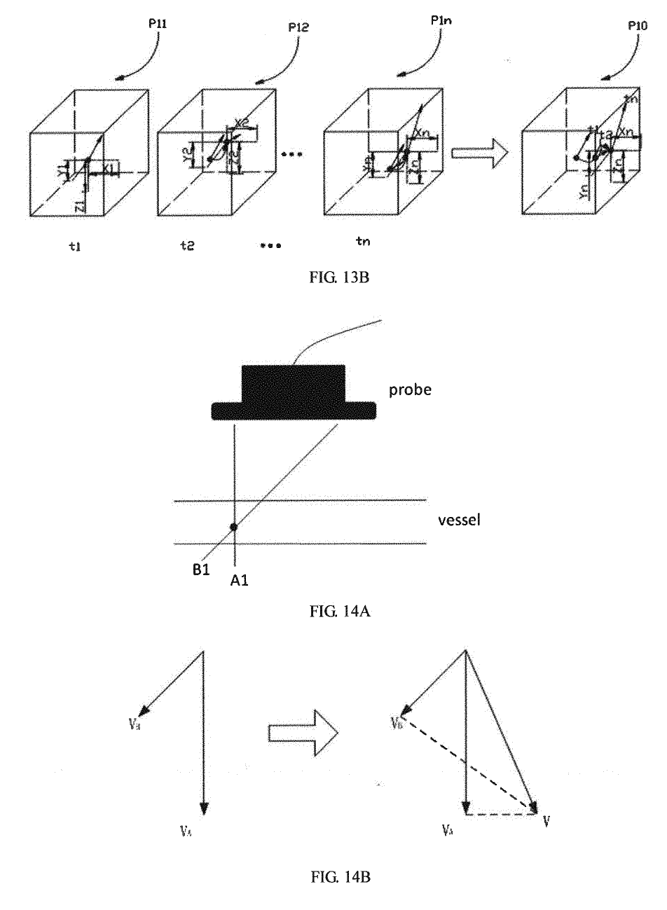

In the case that there are two angles in step S100, the magnitudes and directions of the flow velocities at all position to be measured at one moment may be obtained through 2N transmissions; in the case that there are three angle, 3N transmissions are needed; and so on. In FIG. 14A, two transmissions A1 and B1 with different angles are shown. After 2N transmissions, the velocity at the dot in the figure may be calculated by velocity synthesis. The velocity synthesis is shown in FIG. 14B. In FIG. 14B, V_{A} and V_{B} are the velocity vector components of the target point at the corresponding positions respectively in the two ultrasound propagation directions A1 and B1 in FIG. 14A. The flow velocity vector V of the target point at the corresponding position may be obtained by spatial velocity synthesis. In the case that there are two ultrasound propagation directions, the image data obtained by each transmission may be used repeatedly to calculate the velocity vector component using the Doppler ultrasound imaging method, thereby reducing the time interval between obtaining the magnitudes and directions of the velocities of the flow in entire field one time and another time. The minimum time interval in the case of two ultrasound propagation directions may be the time spent in the 2 transmissions, the minimum time interval in the case of three ultrasound propagation directions may be the time spent in the 3 transmissions, and so on. With the methods above, at each moment, the magnitudes and directions of the flow velocities at all positions in the entire field may be obtained.

In the case that there are at least three ultrasound propagation directions in step S100, the at least three ultrasound propagation directions corresponding to the at least three groups of echo signals used for calculating at least three velocity vector components may not be in a same plane, such that the calculated flow velocity vector is closer to the velocity vector in real three-dimensional space. This condition may be referred to as constraint related to ultrasound propagation direction.

For example, in step S100 above, the volume ultrasound beams may be transmitted to the scanning target in N (3≦N) ultrasound propagation directions; while in step S400, n velocity vector components may be used to calculate the flow velocity vector of the target point at corresponding position each time, where herein 3≦n<N . I.e., in step S100, the volume ultrasound beams may be transmitted to the scanning target in at least three ultrasound propagation directions, where the adjacent at least three ultrasound propagation directions are not in a same plane. Accordingly, in step S400, at least three blood flow velocity vector components of the target point at the corresponding position corresponding to at least three groups of volume echo signals received successively may be respectively calculated, where one velocity vector component of the target point in the scanning target is calculated based on one of the at least three groups of volume echo signals. The flow velocity vector of the target point at the corresponding position may be synthesized based on the velocity vector components in the at least three ultrasound propagation directions.

In order to reduce the amount of calculation and the complexity of the scanning and calculation, it is also possible that in step S100 the volume ultrasound beams are transmitted to the scanning target in N (3≦N) ultrasound propagation directions while in step S400 N velocity vector components are used to calculate the flow velocity vector of the target point at the corresponding position each time. I.e., in step S100, the volume ultrasound beams may be transmitted to the scanning target in at least three ultrasound propagation directions, where the at least three ultrasound propagation directions are not in a same plane. Accordingly, in step S400, the velocity vector components of the target point at the corresponding position in all of the ultrasound propagation directions corresponding to the at least three groups of volume echo signals may be respectively calculated, where one velocity vector component of the target point in the scanning target is calculated based on one of the received at least three groups of volume echo signals. The flow velocity vector of the target point at the corresponding position may be synthesized based on the velocity vector components in all of the ultrasound propagation directions.

In order to satisfy the constraint related to ultrasound propagation direction, both “the adjacent at least three ultrasound propagation directions being not in a same plane” and “the at least three ultrasound propagation directions being not in a same plane” may be implemented by adjusting the time delay of the transducers used for the transmission of the ultrasound beams and/or driving the transducers used for the transmission of the ultrasound beams to steer to change the emission direction of the ultrasound waves in order to obtain different ultrasound propagation directions. Herein, driving the transducers used for the transmission of the ultrasound beams to deflect to change the emission direction of the ultrasound waves may be implemented by, e.g., providing drive control device for each linear probe or each transducer in the probe group arranged in array and adjusting the steering angle or time delay of the probes or transducers in the probe group such that the scanning bodies formed by the volume ultrasound beams transmitted by the probe group have different steering amount, thereby obtaining different ultrasound propagation directions.

In some embodiments, user-selectable items, or user-selectable buttons, may be provided on the display interface, by which the user may select the number of the ultrasound propagation directions or the number of the velocity vector components used for the synthesis of the flow velocity vector in step S400 above, and thereby instruction information may be generated. Based on the instruction information, the number of the ultrasound propagation directions in step S100 above may be adjusted and the number of the velocity vector components used for the synthesis of the flow velocity vector may be determined according to the number of the ultrasound propagation directions, and alternatively the number of the velocity vector components used for the synthesis of the flow velocity vector of the target point at the corresponding position in step S400 may be adjusted, thereby providing more comfortable experience and more flexible information extraction interface for the user.

In step S500, the stereoscopic display device 8 may display the obtained three-dimensional ultrasound image data to form a spatial stereoscopic image of the scanning target and superimpose the flow velocity vector information on the spatial stereoscopic image. In the present disclosure, the spatial stereoscopic image may be displayed in real-time or non-real-time. In the cast that it is displayed in non-real-time, a plurality of frames of three-dimensional ultrasound image data within a period of time may be cached in order to perform image playback control operations, such as slow play or quick play, etc.

In the present embodiment, holographic display techniques or volume three-dimensional display techniques may be used to display the three-dimensional ultrasound image data to form the spatial stereoscopic image of the scanning target and superimpose the flow velocity vector information on the spatial stereoscopic image.

The hologram herein may include traditional hologram (transmission hologram, reflective hologram, image plane hologram, rainbow hologram or synthetic hologram, etc.) and computer generated hologram (CGH). The CGH may float in the air and have a wide color gamut. In the CGH, a mathematical model of the object whose hologram will be generated may be built, and the physical interference of light waves may be replaced by the calculation steps. In each step, the strength graphics of the CGH model may be determined, and may be outputted to a reconfigurable device. This device may re-modulate the light wave information and reconstruct the output. In general, in CGH, the computer may obtain an interference pattern of a computer graphics (virtual object), which will replace the interference process of the light waves of the object in traditional hologram, through calculation. The diffraction process of the hologram reconstruction may not change in principle, but only device which can reconfigure the light wave information is added, thereby achieving the holographic display of different computer static, dynamic graphics.

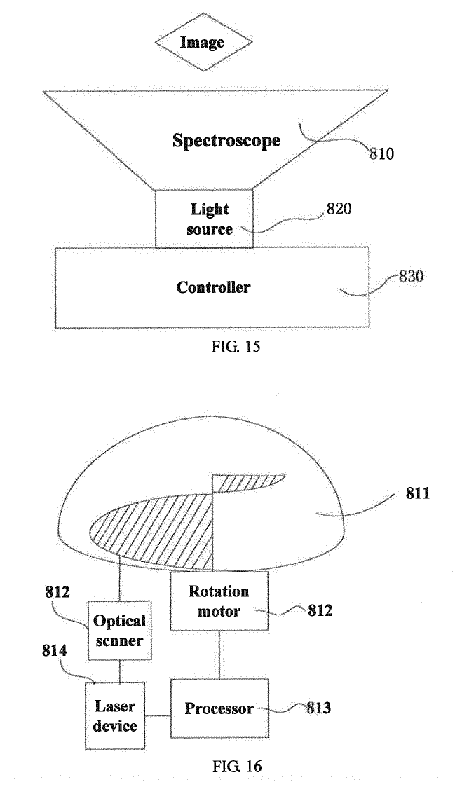

In some embodiments using the holographic display techniques, as shown in FIG. 15, the stereoscopic display device 8 may include a holographic imaging system which may include a light source 820, a controller 830 and a spectroscope 810. The light source 820 may be a spotlight. The controller 830 may include one or more processors, and may receive the three-dimensional ultrasound image data outputted from a data processing unit 9 (or image processing unit 7 therein) through a communication interface, process the image data to obtain the interference pattern of the computer graphics (virtual object), and output the interference pattern to the spectroscope 810. The light irradiated onto the spectroscope 810 by the light source 820 may present the interference pattern to form the spatial stereoscopic image of the scanning target. The spectroscope 810 herein may be special lenses or four-sided pyramid, etc.

As an alternative to the holographic imaging system, the stereoscopic display device 8 may also form the stereoscopic image on air, special lenses, fog screen or the like using a holographic projection device. Accordingly, the stereoscopic display device 8 may also be an air holographic projection device, a laser beam holographic projection device, a holographic projection device with 360 degree holographic display (in which the images are projected to a high-speed rotating minor to obtain the hologram) or a fog screen stereoscopic imaging system, etc.

The air holographic projection device may project the interference pattern of the computer graphics (virtual object) obtained in the embodiments above on an airflow wall to form the spatial stereoscopic image. Since the vibration of the water molecules of the water vapor is not balanced, a hologram with strong three-dimensional sense may be formed. Accordingly, in the present embodiment, a device used for forming the airflow wall may be added based on the embodiment shown in FIG. 15.

The laser beam holographic projection device may use laser beam to project an object. In one embodiment, the laser beam holographic projection device may project the interference pattern of the computer graphics (virtual object) obtained in the embodiments above through laser beams to obtain the spatial stereoscopic image. In the present embodiment, the laser beam projection device may form the hologram through continuous small explosions in the air and the hot substances converted from mixture of oxygen and nitrogen spreading out in the air.

The fog screen stereoscopic imaging system may further include an atomization device based on the embodiment shown in FIG. 15, which may form a water mist wall. The fog screen stereoscopic imaging system may use the water mist wall as the projection screen and project the interference pattern of the computer graphics (virtual object) obtained in the embodiments above on the water mist wall through laser to form the hologram, thereby obtaining the spatial stereoscopic image. The fog screen imaging may form the image in the air through the fine particles in the air using laser. The atomization device may form artificial mist wall which can replace traditional projection screen. Plane fog screen may be formed based on aerodynamics, and projection device may project on the fog screen to form the hologram.

Some holographic display devices have been simply described, and their specific configuration may be similar to related device existing in the market. However, the present disclosure will not be limited to the holographic display devices or systems described above. Other holographic display devices or techniques developed in the future may also be used.

The volume three-dimensional display techniques may form a display object in which the molecular particles are replaced by voxel particles utilizing the special visual mechanism of human. Not only the shape represented by the light waves can be observed, but also the real existence of the voxels can be sensed. The volume three-dimensional display techniques may excite the substances within a transparent display volume and form the voxels utilizing the absorption or scattering of the visible radiation. When the substance within the volume at many directions are excited, the three-dimensional spatial image formed by many voxels dispersed in the three-dimensional space can be obtained. The volume three-dimensional display techniques may include two kinds of techniques below.

(1) Rotating body scanning technique. The rotating body scanning technique may be used for displaying moving object. According to this technique, a series of two dimensional images may be projected to a rotating or moving screen while this screen is moving in a speed which the observer can not perceive. Because of the visual persistence of human, three-dimensional object may be formed by human eye. Therefore, the display system using such stereoscopic displaying techniques can achieve a real three-dimensional display (visible in 360 degree) of the images. In such system, the light beams with different color may be projected on the display media through light deflectors such that the media can present rich colors. Furthermore, such media can enable the light beam to generate discrete visible spots. These spots are voxels and correspond to the points in the three-dimensional image. The groups of voxels may form an image, and the observer can observe this real three-dimensional image from any point of view. The imaging space of the display device using the rotating body scanning techniques may be generated by the rotation or displacement of the screen. The voxels may be activated on the transmission surface when the screen sweeps through the imaging space. The system may include a laser system, a computer control system and a rotation display system, etc.

In some embodiments using the volume three-dimensional display techniques, as shown in FIG. 16, the stereoscopic display device 8 may include a voxel entity part 811, a rotation motor 812, a processor 813, an optical scanner 812 and a laser device 814. The voxel entity part 811 may be a rotating structure in which a rotating surface may be received. The rotating surface may be a spiral surface. The voxel entity part 811 may have media for laser projection display. The processor 813 may control the rotation motor 812 to drive one rotating surface in the voxel entity part 811 to rotate in high speed. And then, the processor 813 may control the laser device to generate R, G and B laser beams, converge the beams into one bunch of chromaticity light and project the chromaticity light on the rotating surface in the voxel entity part 811 to generate a plurality of colored bright spots. When the rotation speed is high enough, a plurality of voxels may be generated in the voxel entity part 811. The group of voxels may form a suspended spatial stereoscopic image.

In other embodiments of the present disclosure, in the configuration shown in FIG. 16, the rotating surface may be an upright projection screen located in the voxel entity part 811. The rotation frequency of this screen may be as high as 730 rpm. The screen may be made of very thin translucent plastic. When a 3D object image needs to be displayed, the processor 813 may first generate, with software, a plurality of section images of the three-dimensional image data (rotating around Z axis and taking a longitudinal section image perpendicular to the X-Y plane every time less than X degree (e.g. 2 degree) are rotated), and project another section image on the upright projection screen every time the upright projection screen is rotated less than X degree. When the upright projection screen is rotated in high speed, the plurality of section images may be in turn projected on the upright projection screen with high speed, thereby forming a natural 3D image which can be observed in all directions.

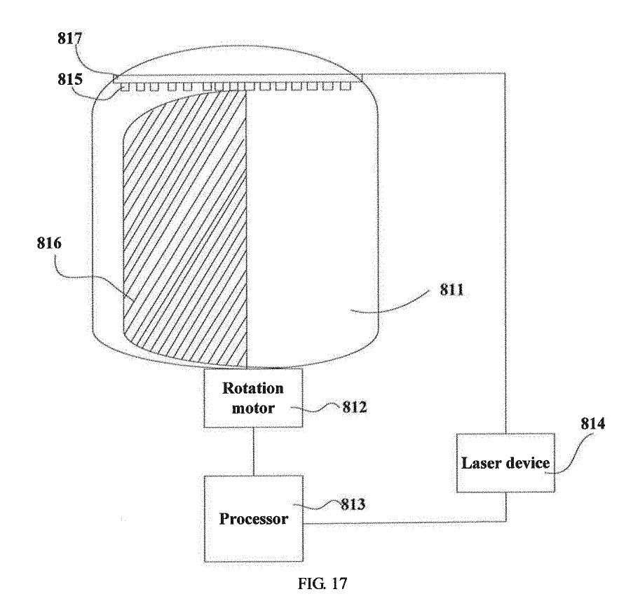

As shown in FIG. 17, the stereoscopic display device 8 may include a voxel entity part 811 having an upright projection screen 816, a rotation motor 812, a processor 813, a laser device 814, and a light emitting array 817. A plurality of beam exits 815 may be arranged on the light emitting array 817. The light emitting array 817 may be three DLP optical chips based on microelectromechanical system (MEMS), each of which may be provided with a high-speed light-emitting array formed by millions of digital micro-mirror devices. The three DLP chips may process R, G and B images, respectively. The R, G, B images may be synthesized into one image. The processor 813 may control the rotation motor 812 to drive the upright projection screen 816 to rotate with high speed. And then, the processor 813 may control the laser device to generate R, G and B laser beams and output the three laser beams to the light emitting array 817. The light emitting array 817 may project the synthetic beam on the upright projection screen 816 being rotated in high speed (where the beams may also be projected on the upright projection screen 816 through the reflection of relay optical lenses) to generate a plurality of voxels for display. The group of the plurality of voxels may form a spatial stereoscopic image suspended in the voxel entity part 811.

(2) Static volume imaging techniques. The static volume imaging techniques may form a three-dimensional stereoscopic image based on frequency conversion techniques. In frequency conversion three-dimensional stereoscopic display, the media in the imaging space may spontaneously emit fluorescence after absorbing multiple photons, thereby generating the visible voxels. The basic principle may be described herein. Two infrared lasers perpendicular to each other may be acted crosswise on the conversion material. After two resonance absorption by the conversion material, the electrons in the emission center may be excited to high excitation level. When the electrons jump to lower level, the emission of visible light may occur. Therefore, one point in the space of the conversion material may be a bright spot which emits light. When the intersection of the two lasers are swept in the three-dimensional space of the conversion material according to a certain trajectory, the path through which the intersection of the two lasers has passed will be a bright band which can emit visible fluorescence, i.e., a three-dimensional stereoscopic graphic which is the same with the movement trajectory of the intersection of the lasers. With this method, a three-dimensional stereoscopic image which can be observed omni-directionally in 360 degree can be observed by naked eye. According to the static volume imaging techniques, display media may be arranged in the voxel entity part 811 in the embodiments above. The media may be formed by a plurality of LCD screens which are arranged with intervals and in a stacked manner (for example, the resolution of each screen may be 1027×748 and the interval between the screens may be about 5 mm). The liquid crystal pixels of these special LCD screens may have special electronic control optical properties. When the voltage is applied to them, the liquid crystal pixel will become parallel to the light beam propagation direction, like the foliages of the blind, such that the light beams irradiating such liquid crystal pixel will pass through. When the voltage applied to them is zero, the liquid crystal pixel will become opaque, thereby diffusely reflecting the irradiating light beams to form a voxel existing in the stacked LCD screens. In this case, the rotation motor in FIG. 16 and FIG. 17 can be omitted. Furthermore, the 3D depth anti-aliasing display techniques may further be used to expand the sense of depth which can be represented by the plurality of LCD screens arranged with intervals therebetween, such that up to 1024×748×608 display resolution can be achieved through 1024×748×20 physical space resolution. As the embodiment shown in FIG. 17, DLP imaging techniques may also be used in the present embodiment.

Some volume three-dimensional display devices have been described above, and their specific configuration may be similar to related device existing in the market. However, the present disclosure will not be limited to the devices or systems based on volume three-dimensional display techniques described above. Other volume three-dimensional display techniques developed in the future may also be used.

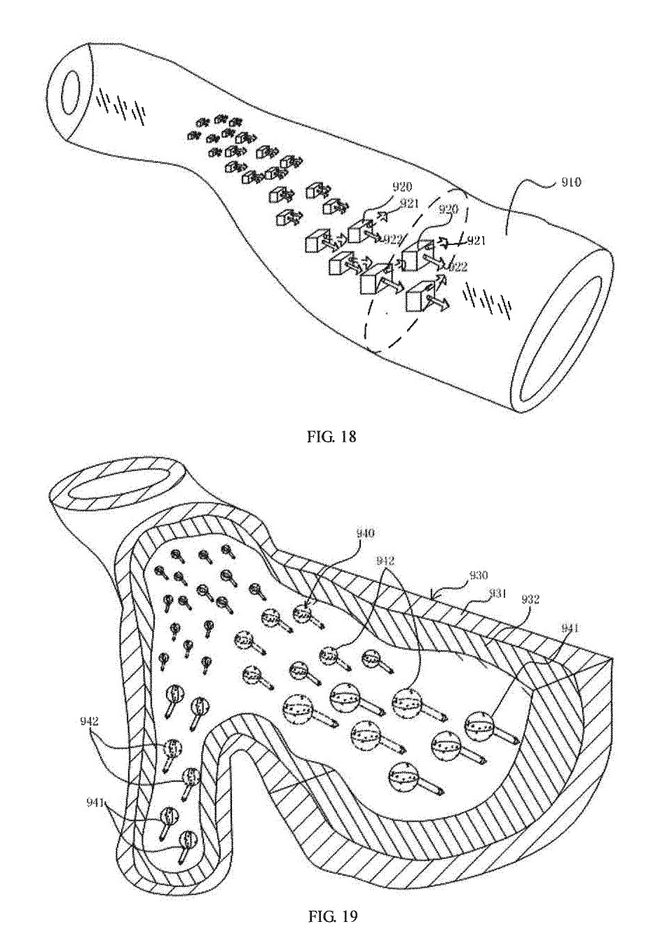

In the present embodiment, the spatial stereoscopic image of the scanning target may be displayed in a certain space or any space, or be represented through the display media such as air, mirrors, fog screens or rotating or resting voxels, etc. Accordingly, In some embodiments, the flow velocity vector information of the target points obtained using the first mode may be superimposed on the spatial stereoscopic image displayed through the methods above, as shown in FIG. 18, where graphics 910 schematically shows a portion of a blood vessel, and the cubes with arrows represent the flow velocity vector information of the target points, in which the direction of the arrow represents the direction of the flow velocity vector of the target point and the length of the arrow represents the magnitude of the flow velocity vector of the target point. In FIG. 18, the solid arrows 922 may represent the flow velocity vector information of the target points at current moment, while the dashed arrows 921 may represent the flow velocity vector information of the target points at a previous moment. In FIG. 18, in order to present three-dimensional display effects, the objects near the observation point are displayed bigger, while the objects far from the observation point are displayed smaller.

In addition, in some embodiments, the flow velocity vector information of the target points obtained using the second mode above may be superimposed on the spatial stereoscopic image displayed using the methods above, i.e., the flow velocity vector information of the target point may include the flow velocity vectors which are accordingly obtained when the target point successively moves to the corresponding positions in the spatial stereoscopic image, and in step S500, the flow velocity vectors correspondingly obtained when the target point successively moves to the corresponding positions may be displayed to form the flow velocity vector mark which flows over time. As shown in FIG. 19, in order to present three-dimensional display effects, the objects near the observation point are displayed bigger, while the objects far from the observation point are displayed smaller. In FIG. 19, the spheres 940 with arrow may be used to represent the flow velocity vector information of the target points, where the direction of the arrow represents the direction of the flow velocity vector of the target point and the length of the arrow represents the magnitude of the flow velocity vector of the target point. The object 930 may represent a section of blood vessel in the spatial stereoscopic image. In FIG. 19, the solid line spheres 941 with arrow may represent the flow velocity vector information of the target point at current moment, while the dashed line spheres 942 with arrow may represent the flow velocity vector information of the target point at previous moment. With the flow velocity vector information of the target points obtained using the second mode above, the marks 940 which flow over time may be represented in the spatial stereoscopic image.

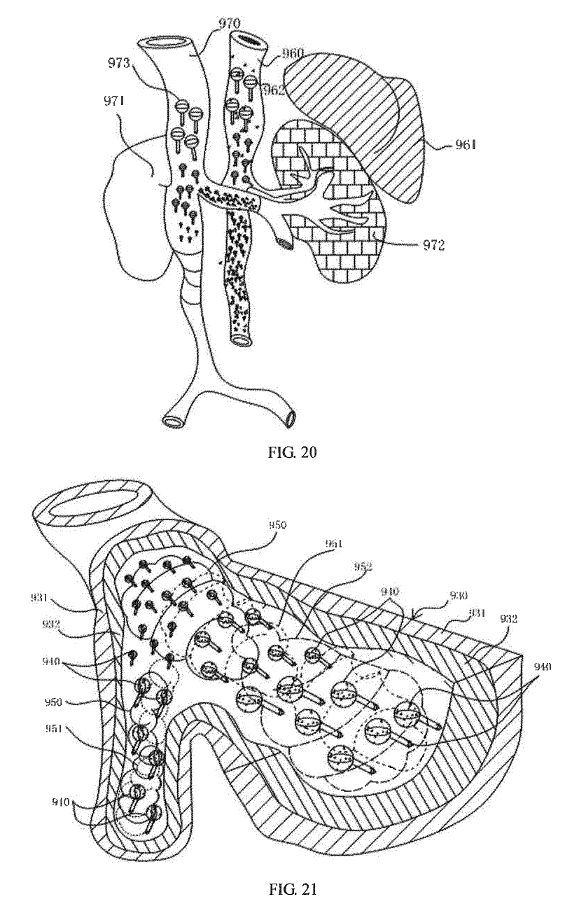

As shown in FIG. 19, the object 930 may represent a section of blood vessel in the spatial stereoscopic image, which may include a first layer of vessel wall 931 and a second layer of vessel wall 932. The two layers of vessel wall may be distinguished using different colors. Furthermore, as shown in FIG. 20, the blood flow velocity vectors of the target points in two groups of blood vessels 960 and 970 may both be represented by spheres 973 and 962 with arrow. In addition, the stereoscopic image regions of other tissue 971, 972 and 961 may be marked with other colors for distinguish. In FIG. 20, these regions may be distinguished by the types of the hatching filled in them. Accordingly, in order to represent three-dimensional imaging effects and distinguish the displayed information, the spatial stereoscopic image may include stereoscopic image regions which represent the tissues according to anatomical tissue structural and hierarchical relationship. These regions may be distinguished from adjacent stereoscopic image region by color parameters.

Furthermore, in order to highlight the flow velocity vector information in the spatial stereoscopic image, the contour lines of the stereoscopic image regions of the tissues may be displayed so as to avoid covering or confusing the flow velocity vector marks. For example, as shown in FIG. 18, the outer contour lines and/or certain section contour lines of a section of blood vessel 910 may be displayed so as to mark the image region in which the flow velocity vector information marks (920) are located, thereby highlighting, and more intuitively and clearly representing, the flow velocity vector marks 920.

As shown in FIG. 18 to FIG. 22, when superimposing the flow velocity vector information on the spatial stereoscopic image in step S500, one or more of the color and shape of the flow velocity vector marks (920, 940, 973, 962, 981, 982) used for representing the flow velocity vector information in the spatial stereoscopic image may be set so as to distinguish them from the background image sections (i.e. the stereoscopic image regions of other tissues in the spatial stereoscopic image, such as blood vessel wall region or lung region, etc.). For example, the blood vessel wall may be displayed as green, while the flow velocity vector marks therein may be displayed as red. Alternatively, the blood vessel wall and the flow velocity vector marks in arteries may be displayed as red, while the blood vessel wall and the flow velocity vector marks in veins may be displayed as green.

Furthermore, one or more of the color and shape of the flow velocity vector marks (920, 940, 973, 962, 981, 982) used for representing the flow velocity vector information in the spatial stereoscopic image may be set so as to distinguish the velocity levels and directions of the displayed flow velocity vector information. For example, the flow velocity vector marks in arteries may use red colors which are gradually changed with respect to each other to represent the different velocity levels, while the flow velocity vector marks in veins may use green colors which are gradually changed with respect to each other to represent the different velocity levels. The deep red color or the deep green color may represent the high velocity, and the light green color or the light red may represent the low velocity. The specific methods for configuring the colors may be those known in the art and will not be described in detail.

In addition, in the embodiments above, the flow velocity vector mark may include the three-dimensional marker with arrow or direction indicator, such as the cube with arrow in FIG. 18 and the sphere with arrow in FIG. 19. The three-dimensional marker may also be a prism with arrow or the cone shown in FIG. 11 and FIG. 12, where the direction of the cone represents the direction of the flow velocity vector; alternatively, the small end of a truncated cone may be used as the direction indicator; alternatively, the direction of the long diagonal of a three-dimensional marker with rhombic longitudinal sections may be used to represent the direction of the flow velocity vector; alternatively, the two ends of the long axis of an ellipsoid may be used as the direction indicator to represent the direction of the flow velocity vector; and so on. The shape of the flow velocity vector marks will not be limited by the present disclosure, and any three-dimensional marker with direction indication may be used to marking the flow velocity vector of the target point. Accordingly, the arrow or the direction indicator of the three-dimensional marker may be used to represent the direction of the flow velocity vector, and the size of the three-dimensional marker may be used to represent the magnitude of the flow velocity vector, so as to more intuitively represent the flow velocity vector information of the target point.

Alternatively, the flow velocity vector mark may also be a three-dimensional marker without arrow or direction indicator, such as the sphere shown in FIG. 12, or other three-dimensional object with any shape such as ellipsoid, cube or cuboid, etc. Accordingly, the rotation speed or size of the three-dimensional marker may be used to represent the magnitude of the flow velocity vector, and the movement of the three-dimensional marker over time may be used to represent the direction of the flow velocity vector, so as to more intuitively represent the flow velocity vector information of the target point. For example, the flow velocity vector of the target point may be calculated using the second mode above, thereby obtaining the flow velocity vector marks flowing over time. The rotation speed or size of the three-dimensional marker may be associated with the magnitude of the flow velocity vector based on level so as to facilitate the marking on the spatial stereoscopic image. The rotation directions of the three-dimensional markers may be the same or different. The rotation speed may be a speed which can be recognized by human eye. Asymmetric three-dimensional markers or three-dimensional markers with signs may be used so as to enable the human eye to observe the rotation of the three-dimensional markers.

Alternatively, the rotation speed of the three-dimensional marker may be used to represent the magnitude of the flow velocity vector, and the direction of the arrow may be used to represent the direction of the flow velocity vector. Accordingly, the present disclosure will not be limited to the methods for representing the magnitude or direction of the flow velocity vector described above. In the present disclosure, the size or rotation speed of the three-dimensional marker used for marking the flow velocity vector of the target point may be used to represent the magnitude of the flow velocity vector and/or the direction of the arrow or indirection indicator of the three-dimensional marker or the movement of the three-dimensional marker over time may be used to represent the direction of the flow velocity vector.

In addition, as shown in FIG. 21, when the enhanced three-dimensional ultrasound image data of at least a part of the scanning target is obtained using grayscale blood flow imaging in step S300 in the embodiments above, the corresponding grayscale characteristics obtained by the grayscale blood flow imaging may also be displayed in the spatial stereoscopic image. For example, whether the enhanced three-dimensional ultrasound image data is processed as a whole as three-dimensional data or respectively as a plurality of two dimensional images, cluster block regions may be obtained in each frame of enhanced three-dimensional ultrasound image data using the methods described below. First, the region of interest representing the flow area may be segmented from one or more frames of enhanced three-dimensional ultrasound image data to obtain the cluster block regions like cloud. In step S500, the cluster block regions like cloud may be displayed in the spatial stereoscopic image to form cluster blocks rolling over time. In FIG. 21, the graphics 950, 951 and 952 drawn with different lines may represent the cluster blocks at different time. It can be seen that the cluster blocks are rolling over time, which vividly represent the rolling of the fluid and provide an omni-directional observation perspective to the observer.

Furthermore, color information may be superimposed on the cluster block regions like cloud so as to more clearly display the cluster blocks. For example, when the blood vessel wall is displayed with red color, white color, or orange-red color may be superimposed on the cluster block regions representing the blood flow for distinguish. Alternatively, in the step of segmenting the region of interest representing the flow area in the enhanced three-dimensional ultrasound image data to obtain the cluster block region like cloud, the region of interest representing the flow area in the enhanced three-dimensional ultrasound image data may be segmented based on the grayscale of image, thereby obtaining the cluster block regions with different grayscale characteristics. For the cluster block regions in the spatial stereoscopic space, the grayscale characteristics herein may be the mean, maximum or minimum of the grayscale values of the spatial points in the whole region, or one or more other values which can represent the grayscale characteristics of the whole region. In the step of displaying the cluster block regions lick cloud in the spatial stereoscopic image, the cluster block regions with different grayscale characteristics may be rendered with different colors. For example, assuming the cluster block regions obtained by the segmentation can be classified into 0-20 class based on the grayscale characteristics, each class may be displayed with one color. Alternatively, the 0-20 class may also be displayed using tints with different purity which belong to a same color, respectively.

As shown in FIG. 24, one cloudy cluster block region 953 may also be segmented based on the grayscale of image to obtain area bodies with different grayscale. The area bodies may be rendered with different colors according to the grayscale thereof. In FIG. 24, different hatchings are filled in different area bodies in the cluster block region 953 in order to represent the rendering with different colors. The methods for rendering may be similar to those in the embodiment above. For example, the area bodies in the cluster block region may be classified into multiple classes based on the grayscale characteristics. Each class may be displayed with one color (or hue), or the multiple classes may be displayed using tints with different purity which belong to a same color, respectively.

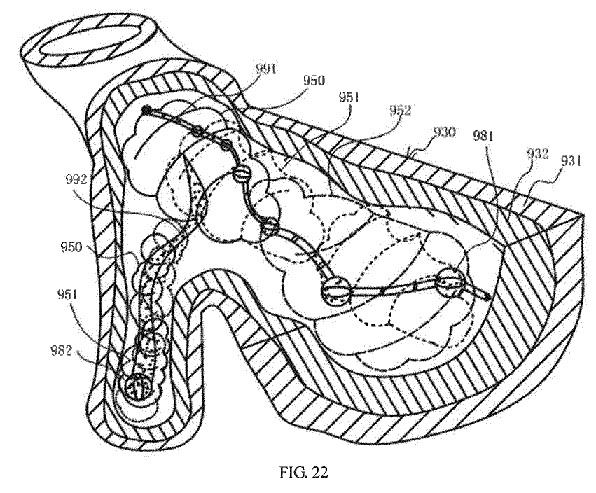

Based on the display of the cloudy cluster block regions described above, another display mode is actually provided. As shown in FIG. 21 and FIG. 22, through the mode switch instruction inputted by the user, the current display mode may be switched to the display mode in which the cloudy cluster block regions are displayed in the spatial stereoscopic image to form the cluster blocks rolling over time.

In some embodiment of the present disclosure, the flow velocity vector information of the target point obtained using the second mode described above may be superimposed on the spatial stereoscopic image displayed using the methods above. The flow velocity vector information of the target point may include the flow velocity vectors correspondingly obtained when the target point successively move to correspondingly positions in the spatial stereoscopic image. In step S500, an connection mark connecting the multiple positions (such as tow or more positions) in the spatial stereoscopic image to which one target point has moved may be formed to represent the movement trajectory of the target point, and be displayed in the spatial stereoscopic image. In FIG. 22, the connection mark used for displaying the movement trajectory may include slender cylinder, segmental slender cylinder or comet tail-like mark, etc. In FIG. 22, the objects near the observation point are displayed bigger, while the objects far from the observation point are displayed smaller, as so to present the three-dimensional display effect. In FIG. 22, the graphics 930 may represent a section of blood vessel in the spatial stereoscopic image. The flow velocity vector marks (the sphere 981 or sphere 982 with arrow) used for marking the blood flow velocity vector information of the target point may successively move, from the initial position of the flow velocity vector mark, to multiple positions of the target point in the spatial stereoscopic image through the slender cylinder or segmental slender cylinder 911 which connects the multiple positions, thereby forming the movement trajectory to facilitate the understanding of the observation to the motion of the target point. In addition, another method for displaying the trajectory is further provided in FIG. 22. For example, certain colors may be displayed in a continuous area where one target point successively moves to multiple positions in the spatial stereoscopic image from the initial position of the flow velocity vector mark to form a comet tail-like mark 992. When the observers observe the movement trajectory of the target point, they will see a flow velocity vector mark 982 followed by a long tail similar to the comet tail.

In some embodiments, in order to facilitate the highlight display of the movement trajectory in the spatial stereoscopic image, the method described above may further include following steps.

First, indication information of the connection mark inputted by the user may be obtained to generate a selection instruction. The indication information may include the shape of the connection mark or the shape and color of the connection line, etc. And then, the parameters of the connection mark used for displaying the movement trajectory in the spatial stereoscopic image may be configured according to the indication information selected by the selection instruction.

In the present disclosure, the color may include any color obtained by adjusting the tint (hue), saturation (purity) or contrast, etc. The connection mark may be implemented in many forms, such as slender cylinder, segmental slender cylinder or comet tail-like mark or any other mark which can represent the direction.

Furthermore, based on the display of the movement trajectory of the target point, another display mode is actually provided by the present disclosure. As shown in FIG. 22, by the mode switch instruction inputted by the user, the current display mode may be switched to displaying the movement trajectory of the target point in the spatial stereoscopic image, i.e. the mode in which the multiple positions in the spatial stereoscopic image to which one target point successively to are connected by the connection mark to form the movement trajectory of the target point.

In addition, the movement trajectory of one or more target points may be displayed, and the initial position may be obtained by inputted instructions. For example, distribution density instruction inputted by the user may be obtained, and the target point may be randomly selected in the scanning target according to the distribution density instruction. Alternatively, the position indication instructions inputted by the user may be obtained, and the target points may be determined according to the position indication instructions.

FIG. 8 schematically shows the flow chart of the ultrasound imaging method of some embodiments of the present disclosure. It should be understood that, although the steps of the flow chart is sequentially shown according to the indication of the arrows in FIG. 8, these steps will not necessarily be performed according to the order indicated by the arrows. Unless expressly stated herein, the performance of these steps will not be limited to a certain order, but may also be in other order. Furthermore, at least a portion of the steps in FIG. 8 may include a plurality of sub-steps or a plurality of stages. The sub-steps or stages will not necessarily be performed at the same moment, but may also be performed at different moments. The sub-steps or stages will not necessarily be performed sequentially, but may also be performed in parallel or alternately with other steps or with at a portion of the sub-steps or stages of other steps.

In the description of the embodiments above, only the implementation of corresponding steps is described. However, as long as there is no contradiction in logic, the embodiments above may be combined with each other to form new technical solutions, which will be still within the scope of the disclosure of the embodiments.

With the description of the embodiments above, a person skilled in the art will understand that the methods described in the embodiments above can be implemented by software and general hardware platforms, or be implemented by hardware. But in many cases, the former may be preferred. Based on this understanding, the essence or the parts contributing to the prior art of the present disclosure may be implemented as software products. The software products may be carried by a nonvolatile computer readable storage media (such as ROM, disk, CD or server cloud), and may include several instructions which, when be executed, can enable a terminal equipment (which may be a mobile phone, a computer, a sever or a network device, etc.) to perform the methods of the embodiments of the present disclosure.

Based on the ultrasound imaging methods described above, the present disclosure may further provide an ultrakmnd imaging system, which may include: a probe 1; a transmitting circuit 2 which may excite the probe to transmitting volume ultrasound beams to the scanning target; a receiving circuit 4 and a beam forming unit 5 which may receive the echoes of the volume ultrasound beams and obtain volume ultrasound echo signals; a data processing unit 9 which may obtain the three-dimensional ultrasound image data of at least a part of the scanning target based on the volume ultrasound echo signals, and may obtain the flow velocity vector information of the target point in the scanning target based on the volume ultrasound echo signals; and a stereoscopic display device 8 which may receive the three-dimensional ultrasound image data and the flow velocity vector information of the target point, display the three-dimensional ultrasound image data to form the spatial stereoscopic image of the scanning target, and display the flow velocity vector information on the spatial stereoscopic image.

The transmitting circuit 2 may perform step S100 above, and the receiving circuit 4 and the beam forming unit 5 may perform S200 above. The data processing unit 9 may include a signal processing unit 6 and/or an image processing unit 7. The signal processing unit 6 may perform the calculation of the velocity vector components and the flow velocity vector information described above, i.e. step S400 above. The image processing unit 7 may perform the image processing processes described above, i.e. step S300 of obtaining the three-dimensional ultrasound image data of at least a part of the scanning target according to the volume ultrasound echo signals obtained in the preset time period. The image processing unit 7 may further output the data including the three-dimensional ultrasound image data and the flow velocity vector information to the stereoscopic display device 8 for display. The performance of the functional units may be similar to the steps of the ultrasound imaging methods described above and will not be described again.

In some embodiments of the present disclosure, the stereoscopic display device 8 may further mark the flow velocity vectors obtained when the target point successively moves to the corresponding positions to form the flow velocity vector mark flowing over time. The specific performance may be similar to those described above.

In some embodiments, the echo signals of the volume plane ultrasound beams may be used to calculate the flow velocity vector components and flow velocity vector information and the three-dimensional ultrasound image data. For example, the transmitting circuit may excite the probe to transmit the volume plane ultrasound beams to the scanning target; the receiving circuit and the beam forming unit may receiving the echoes of the volume plane ultrasound beams and obtain the volume plane ultrasound echo signals; and the data processing unit may obtain the three-dimensional ultrasound image data of at least a part of the scanning target and the flow velocity vector information of the target point according to the volume plane ultrasound echo signals.

Alternatively, the echo signals of the volume plane ultrasound beams may be used to calculate the velocity vector components and the flow velocity vector information, while the echo signals of the volume focused ultrasound beams may be used to obtain the ultrasound images with high quality. Accordingly, the transmitting circuit may excite the probe to transmit the volume focused ultrasound beams to the scanning target; the receiving circuit and the beam forming unit may receive the echoes of the volume focused ultrasound beams and obtain the volume focused ultrasound echo signals; and the data processing unit may obtain the three-dimensional ultrasound image data of at least a part of the scanning target according to the volume focused ultrasound echo signals. Furthermore, the transmitting circuit may excite the probe to transmit the volume plane ultrasound beams to the scanning target, where the transmission of the volume focused ultrasound beams to the scanning target may be inserted between the transmissions of the plane ultrasound beams to the scanning target; the receiving circuit and the beam forming unit may receive the echoes of the volume plane ultrasound beams and obtain the volume plane ultrasound echo signals; and the data processing unit may obtain the flow velocity vector information of the target point in the scanning target according to the volume plane ultrasound echo signals. The alternate transmission of the two kinds of beams may be similar to those described above, and will not be described in detail again.

Furthermore, the data processing unit may further obtain the enhance three-dimensional ultrasound image data of at least a part of the scanning target using the grayscale blood flow imaging according to the volume ultrasound echo signals, and obtain the cluster block regions like cloud by segmenting the region of interest in the enhance three-dimensional ultrasound image data representing the flow area. The stereoscopic display device may further display the cloudy cluster block regions in the displayed spatial stereoscopic image to form the cluster blocks rolling over time. The specific implementation may be similar to those described above.

Alternatively, in some embodiments, as shown in FIG. 1, the system may further include a human-machine interface device 10 which may obtain the instructions inputted by the user. The data processing unit 9 may further perform at least one of following steps: configuring the color parameters of the stereoscopic image regions, which are included in the spatial stereoscopic image and present the tissues according to anatomical tissue structural and hierarchical relationship, according to the instructions inputted by the user; configuring one or more of the color and shape of the flow velocity vector mark which marks the flow velocity vector information in the spatial stereoscopic image according to the instructions inputted by the user; switching to the display mode of displaying the cloudy cluster block regions in the displayed spatial stereoscopic image to form the cluster blocks rolling over time according to the instructions inputted by the user; configuring the color of the cluster block regions according to the instructions inputted by the user; randomly selecting target points in the scanning target according to the distribution density instructions inputted by the user; obtaining the target point according to the position indication instructions inputted by the user; configuring the color and shape of the connection mark according to the instructions inputted by the user, where the stereoscopic display device may further form a movement trajectory of the target point by connecting multiple positions in the ultrasound image to which a same target point successively moves using a connection mark and display the movement trajectory in the spatial stereoscopic image; configuring the position or parameter of the stereoscopic cursor displayed in the spatial stereoscopic image according to the instructions inputted by the user, where the stereoscopic display device may further display the stereoscopic cursor in the spatial stereoscopic image; and switching the types of the volume ultrasound beams transmitted to the scanning target by the probe under the excitation of the transmitting circuit according to the instructions inputted by the user.

The steps performed by the data processing unit 9 according to the instructions inputted by the user may be similar to those described above and will not be described in detail again.

The stereoscopic display device 8 may include one of the holographic display device based on holographic display techniques and voxel display device based on volume three-dimensional techniques. The specific configuration may be similar to those described with respect to S500 above, as shown in FIG. 15 to FIG. 17.

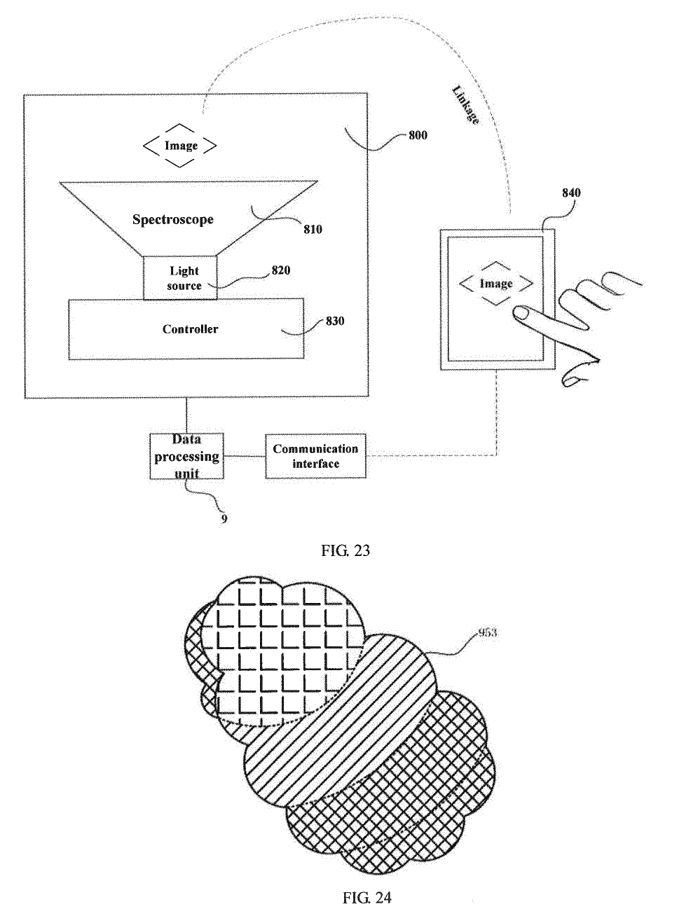

In some embodiment of the present disclosure, the human-machine interface device may include an electronic device 840 which is connected with the data processing unit and provided with a touch screen. The electronic device 840 may be connected with the data processing unit 9 through a communication interface (wireless or wired communication interface) so as to receive the three-dimensional ultrasound image data and the flow velocity vector information of the target point, and display them on the touch screen to present the ultrasound image (which may be two dimensional or three-dimensional ultrasound image based on the three-dimensional ultrasound image data) and the flow velocity vector information superimposed on the ultrasound image. The electronic device 840 may further receive the operation instructions inputted by the user through the touch screen and transfer the operation instructions to the data processing unit 9. The operation instructions herein may include one or more instructions inputted by the user with respect to the data processing unit 9 described above. The data processing unit 9 may obtain related configuration or switch instructions according to the operation instructions and transfer them to the stereoscopic display device 800. The stereoscopic display device 800 may adjust the display of the spatial stereoscopic image according to the configuration or switch instructions so as to synchronously display, in the spatial stereoscopic image, the results of the controls such as image rotation, image parameter configuration, image display mode switch or the like performed according to the operation instructions inputted by the user through the touch screen. As shown in FIG. 23, the stereoscopic display device 800 may be the holographic display device as shown in FIG. 15. Accordingly, by synchronously displaying the ultrasound image and the flow velocity vector information superimposed on the ultrasound image on the electronic device 840 connected with the data processing unit 9, a method for inputting the operation instructions may be provided to the observer, by which the observer may interact with the displayed spatial stereoscopic image.

Furthermore, in some embodiments, the human-machine interface device 10 may also be physical operation key (such as keyboard, operating lever or roller, etc), virtual keyboard or gesture input device with camera, etc. The gesture input device herein may include a device which may acquire the image when the gesture is inputted and track the gesture input using image recognition techniques. For example, the device may use an infrared camera to acquire the image of the gesture input and obtain the operation instructions represented by the gesture input using the image recognition techniques.

Accordingly, the present disclosure provides ultrasound flow imaging methods and ultrasound imaging systems which can, overcoming the drawbacks of existing ultrasound imaging system in displaying the blood flow, be suitable for imaging and displaying the blood flow. The systems may provide better observation perspective to the user through the 3D stereoscopic display techniques. Not only the scanning position can be observed in real time, but also the blood flow information can be presented more realistically by the image. The movement of the fluid in the scanning target may be reproduced really, multiple-angle, omni-directional observation can be provided to the user, and more comprehensive, more accurate image data can be provided to medical personnel. Accordingly, a new display method for blood flow imaging may be created for achieving blood flow display in ultrasound systems. In addition, the present disclosure further provides new methods for calculating the flow velocity vector information of the target point, which can provide more real data regarding the actual flow state of the fluid and intuitively present the movement trajectory of the target point along the flow direction. Furthermore, the present disclosure further provides more personalized custom services, and provides more accurate, more intuitive data support for the user observing the real flow state.

The present disclosure further provides display methods which can present grayscale enhancement effect in the ultrasound stereoscopic image. In the methods, different colors may be used to represent the image of the region of interest with change in grayscale, and the flow state of the cluster block regions may be dynamically presented. Compared with the traditional display, the 3D display of the present disclosure is more vivid and more real, and contains more information.

Several embodiments of the present disclosure have been described above, which is relatively specific and detailed. However, it can not be interpreted as limitation to the scope of the present disclosure. It should be noted that, for a person of ordinary skill in the art, many modifications, improvements and combination can be made without departing from the concepts of the present disclosure, all of which are within the scope of the present disclosure. Therefore, the protection scope of the present disclosure should be determined by the appended claims.

1. An ultrasound flow imaging method, comprising:
transmitting volume ultrasound beams to a scanning target;
receiving echoes of the volume ultrasound beams and obtaining volume ultrasound echo signals;
obtaining three-dimensional ultrasound image data of at least a part of the scanning target based on the volume ultrasound echo signals;
obtaining flow velocity vector information of a target point in the scanning target based on the volume ultrasound echo signals; and
displaying the three-dimensional ultrasound image data to form a spatial stereoscopic image of the scanning target and superimposing the flow velocity vector information in the spatial stereoscopic image.

2. The ultrasound flow imaging method of claim 1, wherein the flow velocity vector information of the target point comprises flow velocity vectors obtained when the target point successively moves to corresponding positions in the spatial stereoscopic image.

3. The ultrasound flow imaging method of claim 2, wherein superimposing the flow velocity vector information on the spatial stereoscopic image further comprises:
marking the flow velocity vectors obtained when the target point successively moves to the corresponding positions to form a flow velocity vector mark flowing over time.

4. The ultrasound flow imaging method of claim 1, wherein, the spatial stereoscopic image comprises at least one of:
stereoscopic image regions which represent tissues according to anatomical tissue structural and hierarchical relationship, wherein color of the stereoscopic image region is configured to distinguish the stereoscopic image region from adjacent stereoscopic image regions; and
a contour line of the stereoscopic image region is displayed so as to highlight the flow velocity vector information of the target point.

5. The ultrasound flow imaging method of claim 1, wherein superimposing the flow velocity vector information on the spatial stereoscopic image comprises configuring one or more of a color and a shape of a flow velocity vector mark used for marking the flow velocity vector information in the spatial stereoscopic image so as to distinguish the flow velocity vector information from a background image section in the spatial stereoscopic image or distinguish a velocity level of the flow velocity vector information.

6. The ultrasound flow imaging method of claim 1, wherein obtaining the three-dimensional ultrasound image data of at least a part of the scanning target further comprises:
obtaining an enhanced three-dimensional ultrasound image data of at least a part of the scanning target using grayscale blood flow imaging.

7. The ultrasound flow imaging method of claim 6, wherein obtaining the three-dimensional ultrasound image data of at least a part of the scanning target further comprises segmenting a region of interest representing a flow area in the enhanced three-dimensional ultrasound image data to obtain a cluster block region like a cloud; and wherein displaying the three-dimensional ultrasound image data to form the spatial stereoscopic image of the scanning target further comprises displaying the cluster block region like a cloud in the displayed spatial stereoscopic image to form a cluster block rolling over time.

8. The ultrasound flow imaging method of claim 7, wherein displaying the cluster block region like a cloud in the displayed spatial stereoscopic image comprises superimposing color on the cluster block region like a cloud.

9. The ultrasound flow imaging method of claim 8, wherein, segmenting the region of interest representing the flow area in the enhanced three-dimensional ultrasound image data to obtain the cluster block region like a cloud comprises segmenting the region of interest representing the flow area in the enhanced three-dimensional ultrasound image data based grayscale of image to obtain the cluster block regions with different grayscale characteristics; and wherein
displaying the cluster block region like a cloud in the displayed spatial stereoscopic image comprises rendering the cluster block regions with different grayscale characteristics with different colors.

10. The ultrasound flow imaging method of claim 8, wherein superimposing the color on the cluster block region like a cloud comprises:
in one cluster block region like a cloud, superimposing different colors on different area bodies in the cluster block region according to grayscale thereof.

11. The ultrasound flow imaging method of claim 1, wherein at least one of size or rotation speed of a three-dimensional marker used for marking the flow velocity vector of the target point is used to represent a magnitude of the flow velocity vector, and wherein at least one of a direction of an arrow on a three-dimensional marker or a direction of a direction indicator or a movement of the three-dimensional marker over time is used to represent a direction of the flow velocity vector.

12. The ultrasound flow imaging method of claim 1, wherein, in obtaining the flow velocity vector information of the target point in the scanning target based on the volume ultrasound echo signals, the target point is selected by at least one of:
obtaining an distribution density instruction inputted by an user and randomly selecting the target point in the scanning target according to the distribution density instruction;
obtaining a position indication instruction inputted by a user and obtaining the target point according to the position indication instruction; or
randomly selecting the target point in the scanning target according to a preset distribution density.

13. The ultrasound flow imaging method of claim 12, wherein, the selection is performed by moving a stereoscopic cursor displayed in the spatial stereoscopic image, or the distribution density or position of the target point is selected by a gesture input so as to obtain the distribution density instruction or position indication instruction inputted by the user.

14. The ultrasound flow imaging method of claim 2, wherein superimposing the flow velocity vector information on the spatial stereoscopic image further comprises:
using a connection mark to connect multiple positions in the spatial stereoscopic image to which a same target point successively moves to form a movement trajectory of such target point, and displaying the connection mark in the spatial stereoscopic image.

15. The ultrasound flow imaging method of claim 14, wherein the connection mark comprises a slender cylinder, a segmental slender cylinder, or a comet tail-like mark.

16. The ultrasound flow imaging method of claim 1, wherein obtaining the flow velocity vector information of the target point in the scanning target based on the volume ultrasound echo signals comprises:
obtaining at least two frames of three-dimensional ultrasound image data based on the volume ultrasound echo signals;
obtaining a gradient in time direction at the target point based on the three-dimensional ultrasound image data, and obtaining a first velocity component in an ultrasound propagation direction at the target point based on the three-dimensional ultrasound image data;
obtaining a second velocity component in a first direction and a third velocity component in a second direction at the target point based on the gradient and the first velocity component, wherein the first direction, the second direction and the ultrasound propagation direction are perpendicular to each other; and
synthesizing the flow velocity vector of the target point using the first velocity component, the second velocity component and the third velocity component.

17. The ultrasound flow imaging method of claim 1, wherein a process from transmitting the volume ultrasound beams to the scanning target to obtaining the three-dimensional ultrasound image data do the flow velocity vector information of the target point comprises:
transmitting volume plane ultrasound beams to the scanning target,
receiving echoes of the volume plane ultrasound beams and obtaining volume plane ultrasound echo signals,
obtaining the three-dimensional ultrasound image data based on the volume plane ultrasound echo signals, and
obtaining the flow velocity vector information of the target point based on the volume plane ultrasound echo signals; or
transmitting volume plane ultrasound beams and volume focused ultrasound beams to the scanning target, respectively,
receiving echoes of the volume plane ultrasound beams and obtaining volume plane ultrasound echo signals,
receiving echoes of the volume focused ultrasound beams and obtaining volume focused ultrasound echo signals,
obtaining the three-dimensional ultrasound image data based on the volume focused ultrasound echo signals, and
obtaining the flow velocity vector information of the target point based on the volume plane ultrasound echo signals.

18. The ultrasound flow imaging method of claim 1, wherein the three-dimensional ultrasound image data is displayed to form the spatial stereoscopic image of the scanning target and the flow velocity vector information is superimposed in the spatial stereoscopic image using holographic display techniques or volume three-dimensional display techniques.

19. The ultrasound flow imaging method of claim 7, further comprising:
obtaining a mode switch instruction inputted by an user and switching from a current display mode of the spatial stereoscopic image to a display mode of displaying the cluster block region like a cloud in the spatial stereoscopic image to form the cluster block rolling over time.

20. The ultrasound flow imaging method of claim 1, wherein transmitting the volume ultrasound beams to the scanning target such that the volume ultrasound beams propagate in a space in which the scanning target is located to form a scanning body comprises:
exciting a portion, or all, of transducers to transmit the volume ultrasound beams to the scanning target in one or more ultrasound propagation directions; or
dividing transducers into multiple transducer regions, and exciting a portion, or all, of the transducer regions to transmit the volume ultrasound beams to the scanning target in one or more ultrasound propagation directions;
wherein each scanning body is derived from the volume ultrasound beams transmitted in one ultrasound propagation direction.

21. The ultrasound flow imaging method of claim 20, wherein receiving the echoes of the volume ultrasound beams and obtaining the volume ultrasound echo signals comprises:
receiving echoes of the volume ultrasound beams from multiple scanning bodies and obtaining multiple groups of volume echo signals;
and wherein obtaining the flow velocity vector information of the target point in the scanning target based on the volume ultrasound echo signals comprise s:
obtaining multiple velocity components based on the multiple groups of volume echo signals, wherein one velocity component of the target point in the scanning target is calculated based on one of the multiple groups of volume echo signals; and
synthesizing the flow velocity vector of the target point using the multiple velocity components, and generating the flow velocity vector information of the target point.

22. An ultrasound flow imaging system, comprising:
a probe;
a transmitting circuit which excites the probe to transmit volume ultrasound beams to a scanning target;
a receiving circuit and a beam forming unit which receive echoes of the volume ultrasound beams and obtain volume ultrasound echo signals;
a data processing unit which obtains three-dimensional ultrasound image data of at least a part of the scanning target based on the volume ultrasound echo signals and obtains flow velocity vector information of a target point in the scanning target based on the volume ultrasound echo signals; and
a stereoscopic display device which receives the three-dimensional ultrasound image data and the flow velocity vector information of the target point, displays the three-dimensional ultrasound image data to form a spatial stereoscopic image of the scanning target, and superimposes the flow velocity vector information in the spatial stereoscopic image.

23. The ultrasound flow imaging system of claim 22, wherein the stereoscopic display device further marks flow velocity vectors obtained when the target point successively moves to corresponding positions to form a flow velocity vector mark flowing over time.

24. The ultrasound flow imaging system of claim 22, wherein, the transmitting excites the probe to transmit volume plane ultrasound beams to the scanning target; the receiving circuit and the beam forming unit receive echoes of the volume plane ultrasound beams and obtain volume plane ultrasound echo signals; and the data process unit further obtains three-dimensional ultrasound image data of at least a part of the scanning target and the flow velocity vector information of the target point based on the volume plane ultrasound echo signals; or
the transmitting circuit excites the probe to transmit volume focused ultrasound beams and volume plane ultrasound beams to the scanning target; the receiving circuit and the beam forming unit receive echoes of the volume focused ultrasound beams and obtain volume focused ultrasound echo signals, and receive echoes of the volume plane ultrasound beams and obtain volume plane ultrasound echo signals; and the data processing unit obtains three-dimensional ultrasound image data of at least a part of the scanning target based on the volume focused ultrasound echo signals and obtains the flow velocity vector information of the target point in the scanning target based on the volume plane ultrasound echo signals.

25. The ultrasound flow imaging system of claim 22, wherein the system uses a connection mark to connect multiple positions in the spatial stereoscopic image to which a same target point successively moves to form a movement trajectory of such target point, and displays the connection mark in the spatial stereoscopic image.

26. The ultrasound flow imaging system of claim 22, wherein the data processing unit obtains enhanced three-dimensional ultrasound image data of at least a part of the scanning target using grayscale blood flow imaging techniques based on the volume ultrasound echo signals.

27. The ultrasound flow imaging system of claim 26, wherein, the data processing unit further segments a region of interest representing a flow area in the enhanced three-dimensional ultrasound image data to obtain a cluster block region like a cloud; and
the stereoscopic display device further displays the cluster block region like a cloud in the displayed spatial stereoscopic image to form a cluster block rolling over time.

28. The ultrasound imaging system of claim 22, further comprising:
a human-machine interface device which obtains an instruction inputted by a user;
wherein the data processing unit further performs at least one of:
configuring a color parameter of a stereoscopic image region, which is included in the spatial stereoscopic image and presents tissues according to anatomical tissue structural and hierarchical relationship, according to the instruction inputted by the user;
configuring one or more of a color and a shape of a flow velocity vector mark which marks the flow velocity vector information in the spatial stereoscopic image according to the instruction inputted by the user;
switching to a display mode of displaying the cluster block region like a cloud in the displayed spatial stereoscopic image to form a cluster block rolling over time according to the instruction inputted by the user;
configuring a color of the cluster block region according to the instruction inputted by the user;
randomly selecting the target point in the scanning target according to a distribution density instruction inputted by the user;
obtaining the target point according to a position indication instruction inputted by the user;
configuring a color and a shape of a connection mark according to the instruction inputted by the user, wherein the stereoscopic display device further forms a movement trajectory of the target point by connecting multiple positions in the ultrasound image to which a same target point successively moves using a connection mark and displays the movement trajectory in the spatial stereoscopic image;
configuring a position or a parameter of a stereoscopic cursor displayed in the spatial stereoscopic image according to the instruction inputted by the user, wherein the stereoscopic display device further displays the stereoscopic cursor in the spatial stereoscopic image; and
switching types of the volume ultrasound beams transmitted to the scanning target by the probe under an excitation of the transmitting circuit according to the instruction inputted by the user.

29. The ultrasound flow imaging system of claim 22, wherein the stereoscopic display device includes one of a holographic display device based on holographic display techniques and a voxel display device based on volume three-dimensional techniques.

30. The ultrasound flow imaging system of claim 22, further comprising a human-machine interface device which obtains an instruction inputted by an user; wherein, the human-machine interface device comprises an electronic device which is connected with the data processing unit and provided with a touch screen;
the electronic device receives the three-dimensional ultrasound image data and the flow velocity vector information of the target point and displays the three-dimensional ultrasound image data and the flow velocity vector information on the touch screen so as to present ultrasound image and the flow velocity vector information superimposed on the ultrasound image, and receives an operation instruction inputted by an user through the touch screen and transfers the operation instruction to the data processing unit;
the data process obtains a related configuration or a switch instruction according to the operation instruction and transfers the related configuration or switch instruction to the stereoscopic display device; and
the stereoscopic display device adjusts display of the spatial stereoscopic image according to the configuration or switch instruction.
